(19) United States
(12) Patent Application Publication
(10) Pub. No.: US 2024/0024361 A1
(43) Pub. Date: Jan. 25, 2024

(54) METHODS AND COMPOSITIONS FOR TREATING MULTIPLE SCLEROSIS

(71) Applicant: THE CHILDREN'S MEDICAL CENTER CORPORATION, Boston, MA (US)

(72) Inventors: Paolo FIORINA, Boston, MA (US); Alessandra BIFFI, Boston, MA (US)

(73) Assignee: THE CHILDREN'S MEDICAL CENTER CORPORATION, Boston, MA (US)

(21) Appl. No.: 18/002,633

(22) PCT Filed: Jun. 25, 2021
(86) PCT No.: PCT/US2021/039077

§ 371 Date: Dec. 20, 2022

Related U.S. Application Data
(60) Provisional application No. 63/044,590, filed on Jun. 26, 2020.

(51) Int. Cl. A61K 35/28; A61P 29/00; C12N 5/0789

(52) U.S. Cl. CPC A61K 35/28; A61P 29/00; C12N 5/0647; A61K 2035/124

(57) ABSTRACT

The disclosed invention provides a method of treating a central nervous system (CNS) disease or disorder comprising administering to a subject diagnosed with the CNS disease or disorder a composition comprising a population of genetically modified, programmed cell death-1 receptor ligand (PD-L1)+-expressing hematopoietic stem cells (HSCs), wherein the CNS disease or disorder involves inflammation of the CNS. In one embodiment, the CNS disease or disorder is Multiple Sclerosis (MS). In certain aspects, the hematopoietic stem cells (HSCs) are obtained from a subject having a CNS disease or disorder prior to modification.

### CROSS-REFERENCE TO RELATED APPLICATION

This application is a 371 National Phase Entry of International Patent Application No. PCT/US2021/039077 filed on Jun. 25, 2021, which is an International Application which designated the U.S., and which claims the benefit of and priority to under 35 U.S.C. § 119(e) of U.S. Provisional Application No. 63/044,590 filed on Jun. 26, 2020, the contents of which are incorporated herein by reference in their entirety.

### FIELD OF THE INVENTION

The field of the invention relates to methods for treating or preventing multiple sclerosis and inflammation of the central nervous system.

### SEQUENCE LISTING

The instant application contains a Sequence Listing which has been submitted electronically in ASCII format and is hereby incorporated by reference in its entirety. Said ASCII copy, created on Dec. 20, 2022, is named 701039-097750WOPT_SL.txt and is 6,417 bytes in size.

### BACKGROUND

Multiple sclerosis (MS) is a central nervous system (“CNS”) disease characterized by an alteration of the immune system that gives rise to abnormal immune responses mediated by auto-reactive and activated T cells inducing chronic inflammatory demyelination in the central nervous system (CNS). Current therapeutic strategies for CNS diseases such as MS do not provide a stable remission of the disease. Therefore, there is need for new therapeutics for treatment of CNS diseases including MS.

### SUMMARY

One aspect provided herein is a method of treating a CNS disease or disorder comprising administering to a subject diagnosed with the CNS disease or disorder a composition comprising a population of genetically modified, programmed cell death-1 receptor ligand (PD-L1)⁺-expressing hematopoietic stem cells (HSCs), wherein the CNS disease or disorder involves inflammation of the CNS.

In one embodiment of any aspect, the CNS disease or disorder is selected from the group consisting of MS, Systemic lupus erythematosus (SLE), inflammatory brain disease, inflammation of the CNS, central nervous system vasculitis, Neuromyelitis Optica Spectrum Disorder.

In one embodiment of any aspect, the CNS disease or disorder is MS.

In various embodiments of any aspect, the MS is relapsing remitting MS (RRMS), secondary progressing MS (SPMS), or primary progressive MS (PPMS). In one embodiment of any aspect, the MS is non-active, active, highly active (HA), or rapidly evolving severe relapsing remitting MS (RE).

In one embodiment of any aspect, the CNS disease or disorder is inflammation of the CNS.

In various embodiments of any aspect, inflammation of the CNS is inflammation of the spinal cord, the brain, or the spinal cord and the brain.

In one embodiment of any aspect, the method further comprises, prior to administering, the step of diagnosing the subject as having CNS disease or disorder.

In one embodiment of any aspect, the method further comprises, prior to administering, the step of receiving the results of an assay that diagnoses a subject as having CNS disease or disorder.

In one embodiment of any aspect, the method further comprises, prior to administering, the step of diagnosing the subject as having MS.

In one embodiment of any aspect, the method further comprises, prior to administering, the step of receiving the results of an assay that diagnoses a subject as having MS.

In one embodiment of any aspect, the method further comprises, prior to administering, the step of diagnosing the subject as having inflammation of the CNS (i.e., neuroinflammation).

In one embodiment of any aspect, the method further comprises, prior to administering, the step of receiving the results of an assay that diagnoses a subject as having inflammation of the CNS.

In one embodiment of any aspect, the PD-L1+-expressing HSCs carry an exogenous copy of a nucleic acid encoding a programmed cell death-1 receptor ligand (PD-L1).

In one embodiment of any aspect, the nucleic acid is a complementary DNA (cDNA). In one embodiment of any aspect, the cDNA has the sequence of SEQ ID NO: 1 or 2.

In one embodiment of any aspect, the nucleic acid is a genomic DNA.

In one embodiment of any aspect, the nucleic acid is integrated into the genome of the cells.

In one embodiment of any aspect, the nucleic acid has been introduced into the cells via a vector. In one embodiment of any aspect, the vector is a viral vector, for example, a lentiviral vector.

In one embodiment of any aspect, the PD-L1⁺-expressing HSCs are mammalian HSCs or human HSCs.

In one embodiment of any aspect, the PD-L1⁺-expressing HSCs are obtained by genetically modifying HSCs obtained from bone marrow, umbilical cord, amniotic fluid, chorionic villi, cord blood, placental blood or peripheral blood.

In one embodiment of any aspect, the PD-L1⁺-expressing HSCs are obtained by genetically modifying HSCs obtained from mobilized peripheral blood.

In one embodiment of any aspect, the method further comprises the step, prior to administering, of obtaining HSCs from bone marrow, umbilical cord, amniotic fluid, chorionic villi, cord blood, placental blood or peripheral blood, and genetically modifying the obtained HSCs by introducing an exogenous copy of a nucleic acid encoding a PD-L1.

In one embodiment of any aspect, the obtained HSC cells are ex vivo cultured before, or after, or both before and after the introduction of the exogenous copy of a nucleic acid encoding a PD-L1.

In one embodiment of any aspect, the HSCs are derived from a healthy individual.

In one embodiment of any aspect, the HSCs are derived from an individual with a diagnosed disease or disorder. In one embodiment of any aspect, the diagnosed disease or disorder is a CNS disease or disorder. In one embodiment of any aspect, the CNS disease or disorder is MS. In one embodiment of any aspect, the CNS disease or disorder is inflammation of the CNS.

In one embodiment of any aspect, the HSCs are derived from the subject. In one embodiment of any aspect, the population of PD-L1+-expressing HSCs are autologous, allogeneic, or xenogeneic to the subject.

In one embodiment of any aspect, the PD-L1⁺-expressing HSCs are produced by a method comprising (a) contacting a population of unmodified HSCs with a vector carrying an exogenous copy of a nucleic acid encoding a PD-L1, thereby obtaining modified HSCs; (b) ex vivo culturing the resultant modified cells from the contacting; and (c) establishing expression of PD-L1 on the modified HSCs, thereby producing a population of modified HSCs cells expressing PD-L1.

In one embodiment of any aspect, the method further comprises establishing that the population of modified HSCs have an at least 1-fold, 2-fold, 3-fold, 4-fold, 5-fold or more percent increase in the number of PD-L1 expressing HSCs compared to the population of unmodified HSCs.

In one embodiment of any aspect, administration is systemic.

In one embodiment of any aspect, administration is local administration to at least a lesion, the brain, or the spinal cord.

In one embodiment of any aspect, the lesion is a site of nerve cell damage. In one embodiment of any aspect, the lesion is present on the brain or spinal cord.

In one embodiment of any aspect, administering reduces, delays, or stops the progression of MS.

In one embodiment of any aspect, administering reduces or eliminates inflammation of the CNS.

In one embodiment of any aspect, administering reduces or eliminates the population of neutrophils in the CNS.

In one embodiment of any aspect, administering increases the population of CD3+ T cells in the CNS.

In one embodiment of any aspect, the PD-L1⁺-expressing HSCs are detected at lesions in the subject following administration.

In one embodiment of any aspect, the method further comprises administering at least one additional therapeutic. In one embodiment of any aspect, the at least one additional therapeutic is an anti-MS therapeutic.

In one embodiment of any aspect, administering is intravenous administration, intrathecal administration, or intracerebroventricular administration.

Another aspect provided herein is a method of treating a CNS disease or disorder associated with inflammation of the CNS in a subject comprising (a) providing a population of unmodified hematopoietic stem cells (HSCs); (b) contacting the population of unmodified HSCs of (a) with a vector carrying an exogenous copy of a nucleic acid encoding a PD-L1, thereby obtaining modified HSCs; (c) ex vivo culturing the resultant modified HSCs from the contacting; (d) establishing expression of PD-L1 on the modified HSCs, thereby producing a population of PD-L1⁺-expressing HSCs; and (e) transplanting said population of PD-L1⁺-expressing HSCs into a recipient subject having a CNS disease or disorder associated with inflammation of the CNS, thereby treating the CNS disease or disorder in the recipient subject.

Another aspect provided herein is a method of treating MS in a subject comprising (a) providing a population of unmodified hematopoietic stem cells (HSCs); (b) contacting the population of unmodified HSCs of (a) with a vector carrying an exogenous copy of a nucleic acid encoding a PD-L1, thereby obtaining modified HSCs; (c) ex vivo culturing the resultant modified HSCs from the contacting; (d) establishing the expression of PD-L1 on the modified HSCs, thereby producing a population of PD-L1⁺-expressing HSCs; and (e) transplanting said population of PD-L1⁺-expressing HSCs into a recipient subject having MS, thereby treating MS in the recipient subject.

Another aspect provided herein is a method of treating or preventing inflammation of the CNS in a subject comprising (a) providing a population of unmodified hematopoietic stem cells (HSCs); (b) contacting the population of unmodified HSCs of (a) with a vector carrying an exogenous copy of a nucleic acid encoding a PD-L1, thereby obtaining modified HSCs; (c) ex vivo culturing the resultant modified HSCs from the contacting; (d) establishing expression of PD-L1 on the modified HSCs, thereby producing a population of PD-L1⁺-expressing HSCs; and (e) transplanting said population of PD-L1⁺-expressing HSCs into a recipient subject having or at risk of having inflammation of the CNS, thereby preventing or treating inflammation of the CNS in the recipient subject.

In one embodiment of any aspect, the population of HSCs autologous to the recipient subject. In one embodiment of any aspect, the population of HSCs allogeneic to the recipient subject. In one embodiment of any aspect, the population of HSCs is xenogeneic to the recipient subject.

Another aspect provided herein is a method of treating a CNS disease or disorder associated with inflammation of the CNS in a subject comprising (a) receiving a population of PD-L1⁺-expressing HSCs; and (b) transplanting said population of PD-L1⁺-expressing HSCs into a recipient subject having a CNS disease or disorder associated with inflammation of the CNS, thereby treating the CNS disease or disorder in the recipient subject.

Another aspect provided herein is a method of treating MS in a subject comprising (a) receiving a population of PD-L1⁺-expressing HSCs; and (b) transplanting said population of PD-L1⁺-expressing HSCs into a recipient subject having MS, thereby treating MS in the recipient subject.

Another aspect provided herein is a method of treating or preventing inflammation of the CNS in a subject comprising (a) receiving a population of PD-L1⁺-expressing HSCs; and (b) transplanting said population of PD-L1⁺-expressing HSCs into a recipient subject having or at risk of having inflammation of the CNS, thereby preventing or treating inflammation of the CNS in the recipient subject.

In one embodiment of any aspect, the population of HSCs autologous to the recipient subject. In one embodiment of any aspect, the population of HSCs allogeneic to the recipient subject. In one embodiment of any aspect, the population of HSCs is xenogeneic to the recipient subject.

Another aspect provided herein is a population of PD-L1⁺-expressing HSCs, comprising HSCs obtained from a subject diagnosed with a CNS disease or disorder and an exogenous copy of a nucleic acid encoding PD-L1.

In one embodiment of any aspect, the exogenous copy of the nucleic acid encoding PD-L1 is integrated into a genome of the HSCs.

Another aspect provided herein is a pharmaceutical composition comprising the any of the populations of PD-L1⁺-expressing HSCs described herein in a physiologically acceptable excipient.

Another aspect provided herein is an ex vivo method of producing a population of PD-L1⁺-expressing hematopoietic stem cells (HSCs), the method comprising: (a) obtaining a population of unmodified HSCs from a subject diagnosed with a CNS disease or disorder; (b) contacting the population of unmodified HSCs with a vector carrying an exogenous copy of a nucleic acid encoding PD-L1, thereby obtaining modified HSCs; (c) ex vivo culturing the resultant modified HSCs; and (d) establishing expression of PD-L1 on the modified HSCs, thereby producing a population of modified HSCs expressing PD-L1.

In one embodiment of any aspect, in step a), the population of unmodified HSCs are obtained from the bone marrow, umbilical cord, amniotic fluid, chorionic villi, cord blood, placental blood or peripheral blood of the subject.

### Definitions

Unless otherwise explained, all technical and scientific terms used herein have the same meaning as commonly understood by one of ordinary skill in the art to which this disclosure belongs. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims.

Definitions of common terms in molecular biology can be found in The Merck Manual of Diagnosis and Therapy, 19th Edition, published by Merck Sharp & Dohme Corp., 2011 (ISBN 978-0-911910-19-3), (2015 digital online edition at merckmanuals.com), Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties. Further, unless otherwise required by context, singular terms shall include pluralities and plural terms shall include the singular.

Unless otherwise stated, the present disclosure was performed using standard procedures known to one skilled in the art, for example, in Michael R. Green and Joseph Sambrook, Molecular Cloning: A Laboratory Manual, Cold Spring Harbor Laboratory Press, Cold Spring Harbor, N.Y., USA (2012); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; edition (2005), Animal Cell Culture Methods (Methods in Cell Biology, Vol. 57, Jennie P. Mather and David Barnes editors, Academic Press, 1st edition, 1998), Methods in Molecular biology, Vol. 180, Transgenesis Techniques by Alan R. Clark editor, second edition, 2002, Humana Press, and Methods in Molecular Biology, Vo. 203, 2003, Transgenic Mouse, editored by Marten H. Hofker and Jan van Deursen, which are all herein incorporated by reference in their entireties.

In some embodiments, as used herein, the term “genetically engineered,” “genetically modified” or “modified” refers to the addition, deletion, or modification of the genetic material in a cell. In some embodiments, the terms, “genetically modified cells” and “modified cells,” are used interchangeably. In one embodiment, “modified HSCs” refer HSCs that carry exogenous copies of a nucleic acid encoding a PD-L1.

In one embodiment, the term “non-modified HSCs” refers to HSCs that do not carry exogenous copies of a nucleic acid encoding a PD-L1.

As used herein, the term “exogenous copy” in the context of a coding nucleic acid refers to an extra copy of the coding nucleic acid that is not the original copy of the gene found in the genome of the HSCs. The extra copy of the coding nucleic acid is typically introduced into the cells. For example, the extra copy is carried in a vector. The extra copy may be integrated into the genome of the cells.

As used herein, the term “cDNA” refers to complementary DNA that is double-stranded DNA synthesized from a messenger RNA (mRNA) template in a reaction catalyzed by the enzyme reverse transcriptase. The cDNA lacks introns.

As used herein, a “genomic DNA” encoding a PD-L1 is to mean the copy of the gene as found in the genome of a cell. The genomic DNA encoding a PD-L1 would include introns and other regulatory sequences in addition to the coding exons.

As used herein, the term “integrated” when used in the context of the nucleic acid encoding a gene, e.g., PD-L1, means that the nucleic acid is inserted into the genome or the genomic sequences of a cell. When integrated, the integrated nucleic acid is replicated and divided into the daughter dividing cells in the same manner as the original genome of the cell.

As used herein, the term “viral vector” is used according to its art-recognized meaning. It refers to a nucleic acid vector construct that includes at least one element of viral origin and may be packaged into a viral vector particle. The vector may be utilized for the purpose of transferring DNA, RNA or other nucleic acids into cells either in vitro or in vivo. Numerous forms of viral vectors are known in the art.

As used herein, the term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes, i.e., T-cells.

As used herein, the term “lentiviral vector” refers to a vector having a nucleic acid vector construct that includes at least one element of lentivirus origin. Lentiviral vectors of the disclosure include, but are not limited to, human immunodeficiency virus (e.g., HIV-1, HIV-2), feline immunodeficiency virus (FIV), simian immunodeficiency virus (SIV), bovine immunodeficiency virus (BIV), and equine infectious anemia virus (EIAV). These vectors can be constructed and engineered using art-recognized techniques to increase their safety for use in therapy and to include suitable expression elements and therapeutic genes.

As used herein, the term “hematopoietic stem cell” or “HSC” refers to a stem cell that is capable of giving rise to all the blood cell types of the three hematopoietic lineages, erythroid, lymphoid, and myeloid. These cell types include the myeloid (monocytes and macrophages, neutrophils, basophils, eosinophils, erythrocytes, megakaryocytes/platelets, dendritic cells), and the lymphoid lineages (T-cells, B-cells, NK-cells).

A “cell of the erythroid lineage” refers to a cell being contacted is a cell that undergoes erythropoeisis such that upon final differentiation it forms an erythrocyte or red blood cell (RBC). Such cells belong to one of three lineages, erythroid, lymphoid, and myeloid, originating from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, cells of the “erythroid lineage,” as the term is used herein, comprise hematopoietic progenitor cells, rubriblasts, prorubricytes, erythroblasts, metarubricytes, reticulocytes, and erythrocytes.

As used herein, “Autologous” refers to cells from the same subject, e.g., “self”.

As used herein, “Allogeneic” refers to cells of the same species that differ genetically to the cell in comparison.

As used herein, “Xenogeneic” refers to cells of a different species to the cell in comparison.

As used herein, “Syngeneic” refers to cells of a different subject that are genetically identical to the cell in comparison.

As used herein, the term “central nervous system disease or disorder” is intended to include a disease, disorder, or condition which directly or indirectly affects the normal functioning or anatomy of a subject's central nervous system.

A “subject,” as used herein, includes any animal that possess a hematopoietic system, an immune system and HSCs. As used herein, a “subject” or “patient” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox or wolf. In some embodiments, the subject is a mammal, e.g., a primate, e.g., a human. The terms, “individual,” “patient” and “subject” are used interchangeably herein. The subject is preferably a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like). A subject as the term is used herein can refer to a male or a female.

A subject can be one that has been previously diagnosed as having, or being at risk for have a disease or order to be treated, e.g., a CNS disease or disorder involving inflammation of the CNS, such as MS. Alternatively, a subject can be one that has not been previously diagnosed as having, or being at risk for have a disease or order to be treated, e.g., MS. A subject can have previously received another therapeutic for the disease or disorder to be treated, or one that is currently undergoing treatment for the disease or disorder to be treated (e.g., the subject can have previously received, or is currently receiving an anti-MS therapeutic).

In one embodiment, as used herein “treatment” or “treating,” includes any beneficial or desirable effect on the symptoms or pathology of a disease or pathological condition, and may include even minimal reductions in one or more measurable markers of the disease or condition being treated, e.g., MS. In another embodiment, treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein “preventing” or “prevention” refers to any methodology where the disease state or disorder (e.g., MS) does not occur due to the actions of the methodology (such as, for example, administration of a composition described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who is not administered a composition described herein).

As used herein, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration. “Pharmaceutically acceptable carriers” exclude tissue culture medium, but can include serum or plasma. The serum or plasma can be derived from human or the subject recipient.

With regard to genetically modifying an HSC, “effective amount” means an amount of biologically active vector particles sufficient to provide successful transduction of cells with the exogenous nucleic acid or to provide successful stimulation of PD-L1 expression in the cell respectively.

As used herein, the term “administering,” refers to the placement of the HSCs described herein or the composition thereof into a recipient subject by a method or route which results in at least partial localization of the HSCs at a desired site, and/or results in the proliferation, engraftment and/or differentiation of the HSCs to PD-L1-expressing progeny cells. The HSCs, or the composition comprising the HSCs can be administered by any appropriate route which results in an effective treatment in the subject.

As used herein, the term “lesion” refers to an area of damage or scarring (sclerosis) in the central nervous system resulting from MS. “Lesions” are also referred to as plaques, and are caused by inflammation that results from the immune system attacking the myelin sheath around nerves.

The term “optional” or “optionally” means that the subsequent described event, circumstance or substituent may or may not occur, and that the description includes instances where the event or circumstance occurs and instances where it does not.

As used herein, a “reference level” refers to a normal, otherwise unaffected cell population, e.g., an HSC cell population, or tissue (e.g., a biological sample obtained from a healthy subject, or a biological sample obtained from the subject at a prior time point, e.g., a biological sample obtained from a patient prior to being diagnosed with a disease or disorder, or a biological sample) that has not been modified using methods disclosed herein.

As used herein, an “appropriate control” refers to an otherwise identical cell (e.g., an HSC cell) population (e.g., obtained from a patient who was not administered a compositions described herein, as compared to a non-control cell) that is untreated, e.g., does not receive the treatments described herein, or received only a subset of the treatments described herein.

The term “statistically significant” or “significantly” refers to statistical significance and generally means a two standard deviation (2SD) or greater difference.

The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other terms are defined within the description of the various aspects and embodiments of the technology of the following.

### BRIEF DESCRIPTION OF THE DRAWINGS

FIGS. 1A and 1B show PD-L1 expression of transduced HSPCs. (FIG. 1A) Flow cytometry plot showing endogenous PD-L1 in untransduced KL cells and LV-induced PD-L1 expression in transduced KL cells (LvPDL1 KL). Isotype control antibody was used as negative control of PD-L1 expression. (FIG. 1B) Quantification PD-L1 expression in untransduced and transduced KL cells; ***P<0.001.

FIG. 2 shows clinical score of EAE-MOG immunized mice treated with LvPDL KL (n=7) or left untreated (n=5); ***P<0.001 for all time points assessed from day 15 onwards.

FIG. 3 shows body weight changes over time. Unlike in untreated mice, body weight values come back to baseline levels in mice treated with LvPDL1 KL, *P<0.05.

FIG. 4 shows detection of CD415.1+ (donor) and CD415.2+ (recipient) cells in the brain and spinal cord in control mice and in mice treated with LvPDL1 KL. Analyses have been performed at sacrifice, i.e. at day 30; *P<0.05, ***P<0.001.

FIG. 5 shows effect of MOG35-55 immunization in the CNS myeloid compartment. The analysis shows no difference among groups, except for an increase in the proportion of CD45high macrophages in the EAE groups, in both brain and spinal cord; *P<0.05.

FIG. 6 shows effect of MOG35-55 immunization on CNS total T cell population. The analysis shows an increase in CD3-f T cells in both brain and spinal cord of EAE mice receiving LvPDL1 KL.

FIG. 7 shows phenotypic effects of the transplantation of PD-L1 transduced HSPCs in EAE-MOG immunized mice, Importantly, the proportion of neutrophils in the brain in EAE mice receiving LvPDL1 KL is comparable to that observed in naïve animals, suggesting the ability of LvPDL1 KL to significantly prevent the extent of neuroinflammation observed in EAE mice left untreated. *P<0.05, **P<0.01, ***P<0.001, ****P<0.0001.

FIG. 8 shows representative flow cytometry plots of the brain from EAE mice receiving treatment with LvPDL1 KL, outlining the gating strategy utilized for samples acquisition and analysis.

FIG. 9 shows a schematic representation of Protocol A and Protocol B showing timing of MOG₃₅₋₅₅ immunization (MOG), Pertussis toxin (PTX) injection and HSPC transplant for the in vivo studies.

FIG. 10 shows representative flow cytometry plots showing the percentage of positive and negative cells for Lineage markers (APC-labelled) and human PD-L1 expression after 24 hours of cell culture.

FIG. 11A shows mean±SEM percent changes in body weight over baseline (day 0) during the study period. FIG. 11A shows mean±SEM clinical scores from a total of N≥20 mice per group during the study period.

FIGS. 12A and 12B show effect of MOG₃₅₋₅₅-immunization and HSPC transplant on myeloid and T cell population in the brain (FIG. 12A) and in the spinal cords (FIG. 12B). Proportions of CD45^{high} inflammatory and CD3⁺ T cells in naïve and MOG₃₅₋₅₅-immunized animals, the latter either receiving (IV PD-L1 HSPCs) or not receiving (Untreated) HSPCs transduced to express PD-L1. *P<0.05, **P<0.01, ***P<0.001, ****P<0.0001.

FIG. 13A shows mean±SEM percent changes in body weight over baseline (day 0) during the study period. FIG. 13B mean±SEM clinical scores in the different groups during the study period. *P<0.05.

FIG. 14A shows mean±SEM percent changes in body weight over baseline (day 0) during the study period. FIG. 14B mean±SEM clinical scores in the different groups during the study period. *P<0.05.

FIG. 15 shows effect of MOG₃₅₋₅₅-immunization and HSPC transplant on myeloid and T cell populations in the brain. Proportions of CD45^{high} inflammatory and CD3⁺ T cells in naïve and MOG₃₅₋₅₅-immunized animals, the latter either not receiving (Untreated) or receiving HSPCs transduced to express PD-L1 by different routes of administration, including ICV and ITL (intrathecal lumbar). *P<0.05, **P<0.01, ***P<0.001.

FIG. 16 shows representative immunofluorescence staining of spinal cord coronal cryosections from naïve and MOG₃₅₋₅₅-immunized animals, the latter either not receiving (untreated) or receiving HSPCs transduced to express PD-L1 by different routes of administration, including IV, ITL and ICV. Upper panels show myelin staining (FluoroMyelin), middle panels nuclear staining (DAPI), and lower panels merge of myelin and DAPI staining. Dotted circles and arrows show areas with loss of signal for myelin along with increased cell density.

### DETAILED DESCRIPTION

Data provided herein show that transplantation of a population of genetically modified, programmed cell death-1 receptor ligand (PD-L1)⁺-expressing hematopoietic stem cells (HSCs) in a standard mouse model of multiple sclerosis—the experimental autoimmune encephalomyelitis (EAE) model—greatly reduced or slowed the disease manifestation and progression and contributed to reprogramming the autoreactive immune response into a de novo self-tolerant immune repertoire. Provided herein in the Examples are data showing that a single intravenous administration of genetically modified PD-L1⁺-expressing HSPCs (i.e., LvPDL1) significantly reduces the clinical disease burden (i.e., neuroinflammation). These observed effects are coupled to a lack in the increase of neutrophil CNS-infiltrating cells, indicating that administrations prevented neuroinflammation.

Accordingly, provided herein are methods of treating a CNS disease or disorder comprising administering to a subject diagnosed with the CNS disease or disorder a composition comprising a population of genetically modified, programmed cell death-1 receptor ligand (PD-L1)⁺-expressing hematopoietic stem cells (HSCs), wherein the CNS disease or disorder involves inflammation of the CNS (i.e., neuroinflammation).

### CNS Disease or Disorder

Various aspects of the present invention relate to methods of treating, preventing, or slowing the progression of a CNS disease or disorder that involves inflammation of the CNS (i.e., neuroinflammation). Exemplary CNS disease or disorders that involve inflammation of the CNS and that can be treated with the methods described herein include, but are not limited to, multiple sclerosis (MS), Systemic lupus erythematosus (SLE), inflammatory brain disease, central nervous system vasculitis, Neuromyelitis Optica Spectrum Disorder.

A CNS disease or disorder is generally characterized as a disease or disorder that directly or indirectly affects the normal functioning or anatomy of a subject's central nervous system.

In one embodiment, the CNS disease or disorder is an autoimmune disease or disorder of the CNS with involvement of pro-inflammatory CD4+ T cells. These T cells are responsible for the release of inflammatory, Th1 type cytokines. Cytokines characterized as Th1 type include interleukin 2 (IL-2), γ-interferon, TNFα and IL-12. Such pro-inflammatory cytokines act to stimulate the immune response, in many cases resulting in the destruction of autologous tissue. Cytokines associated with suppression of T cell response are the Th2 type, and include IL-10, IL-4 and TGF-β. It has been found that Th1 and Th2 type T cells may use the identical antigen receptor in response to an immunogen; in the former producing a stimulatory response and in the latter a suppressive response.

Neuroinflammation

Neuroinflammation is defined as the activation of the brain's innate immune system in response to an inflammatory challenge and is characterized, e.g., by a host of cellular and molecular changes within the brain. Neuroinflammation is widely regarded as chronic, as opposed to acute, inflammation of the central nervous system. Acute inflammation usually follows injury to the central nervous system immediately, and is characterized by inflammatory molecules, endothelial cell activation, platelet deposition, and tissue edema. Chronic inflammation, i.e., neuroinflammation, is the sustained activation of glial cells and recruitment of other immune cells into the brain. This type of neuroinflammation is typically associated with neurodegenerative diseases. Common causes of neuroinflammation include, but are not limited to, toxic metabolites, autoimmunity, aging, microbes or viruses, traumatic brain injury, spinal cord injury, air pollution, passive or second hand smoke exposure.

Neuroinflammatory disorders are defined as conditions associated with damage to components of the nervous system caused by the immune response. Exemplary neuroinflammatory disorders include Alzheimer's disease, Parkinson's disease, amyotrophic lateral sclerosis, acute disseminated encephalomyelitis, acute optic neuritis, transverse myelitis, neuromyelitis optica spectrum disorder, brain injury, autoimmune encephalitis, anti-NMDA receptor encephalitis, Rasmussen's encephalitis, acute necrotizing encephalopathy of childhood, opsoclonus-myoclonus-ataxia syndrome, GLUT-1 deficiency syndrome, etc.

In one the embodiment, the subject has been diagnosed as having neuroinflammation, or is at risk of developing neuroinflammation.

In one embodiment, the subject has been diagnosed as having neuroinflammatory disease or disorder, or is at risk of developing neuroinflammatory disease or disorder.

In one embodiment, the subject is identified or diagnosed as not having acute inflammation of the CNS. In one embodiment, the subject is identified or diagnosed as not being at risk of having acute inflammation of the CNS.

Multiple Sclerosis

Various aspects of the present invention relate to methods of treating, preventing, or slowing the progression of multiple sclerosis (MS). MS is an inflammatory and secondary degenerative disease of the human central nervous system (CNS). The disease affects approximately 300,000 people in the United States and is a disease of young adults, with 70%-80% of diagnoses occurring in subjects between 20 and 40 years of age (Anderson et al. Ann Neurology 31 (3): 333-6 (1992); Noonan et al. Neurology 58: 136-8 (2002)). MS is a heterogeneous disease based on clinical symptoms, magnetic resonance imaging (MRI) scan assessment, and pathological analysis of biopsy and autopsy materials (Lucchinetti et al. Ann Neurol 47: 707-17 (2000)). The disease manifests in various combinations of defects to, e.g., the spinal cord, brain stem, cranial nerve, cerebellar, cerebral, and cognitive syndrome. MS can result in progressive disability in most patients, especially when including the 25-year view. Half of MS patients require a cane to walk within 15 years of disease onset.

MS is a leading cause of neurological disability in young and middle-aged adults, and until the last decade, no beneficial treatment has been known. MS is difficult to diagnose due to nonspecific clinical findings leading to the development of highly structured diagnostic criteria including several advanced technologies consisting of MRI scans, evoked potentials, and cerebrospinal fluid (CSF) studies. All diagnostic criteria for MS depend heavily on the presence and reoccurrence of lesions, for example in the brain or spinal cord, that are not explained by other etiologies such as infection, vascular disorders, or autoimmune diseases (McDonald et al. Ann Neurol 50: 121-7 (2001)).

MS has four disease classifications: (1) relapsing-remitting MS (RRMS; 75%-85% of onset cases), characterized by temporary periods called relapses, flare-ups or exacerbations, when new symptoms appear. Relapses typically last a few days or weeks. At other times, the symptoms seem to disappear, resulting in a remission, or remittance periods; (2) primary progressive MS (PPMS; 10%-15% of onset), characterized by slowly worsening symptoms from the beginning, with no relapses or remissions; (3) progressive relapse Type MS (PRMS; 5% of onset) characterized by steadily worsening disease state from the beginning, with acute relapses but no remissions; and (4) secondary progressive MS (SPMS) (Kremenchutzky et al. Brain 122 (Pt 10): 1941-50 (1999); Confavreux et al. N Engl J Med 343 (20): 1430-8 (2000)) characterized by worsening symptoms more steadily over time, with or without the occurrence of relapses and remissions.

In one embodiment, the MS is relapsing remitting MS (RRMS). In one the embodiment, the subject has been diagnosed as having RRMS, or is at risk of developing RRMS.

In one embodiment, the MS is secondary progressing MS (SPMS). In one the embodiment, the subject has been diagnosed as having SPMS, or is at risk of developing SPMS.

In one embodiment, the MS is primary progressive MS (PPMS). In one the embodiment, the subject has been diagnosed as having PPMS, or is at risk of developing PPMS.

In one embodiment, the MS is progressive relapsing MS (PRMS). In one the embodiment, the subject has been diagnosed as having PRMS, or is at risk of developing PRMS.

In one embodiment, administering a composition described herein to a subject having any classifications of MS prevents the subject from developing at least a second classification of MS. For example, administering the composition to a subject having RRMS will prevent the subject from further developing SPMS.

In one embodiment, administering a composition described herein to a subject having any classifications of MS prevents the subject from developing at least a third classification of MS.

An estimated 50% of patients with RRMS will develop SPMS in 10 years, and up to 90% of patients with RRMS will eventually develop SPMS (Weinshenker et al. Brain 112 (Pt 1): 133-46 (1989)). In one embodiment, administering a composition described herein to a subject having any of the classifications of MS slows the development of a second classification of MS. For example, administering the composition to a subject having RRMS will prevent the subject from further developing SPMS within 10, 11, 12, 13, 14, 15 or more years.

MS can be further classified as non-active, active, highly-active (HA), or rapidly evolving severe relapsing remitting MS (RE). “Active” MS refers to a relapsed state and can be determined based on, e.g., the presence of new symptoms and/or lesions. “Highly active (HA)” MS and “rapidly evolving severe relapsing remitting MS (RE)” refers to the accelerated rate of new symptoms and lesions in a relapsed state. “Non-active” MS refers to a remittance state and can be characterized by the absence of symptoms and/or new lesions.

In one embodiment, the MS is non-active. In one the embodiment, the subject has been diagnosed as having non-active MS, or is at risk of developing non-active MS.

In one embodiment, the MS is active. In one the embodiment, the subject has been diagnosed as having active MS, or is at risk of developing active MS.

In one embodiment, the MS is HA. In one the embodiment, the subject has been diagnosed as having HA MS, or is at risk of developing HA MS.

In one embodiment, the MS rapidly evolving severe relapsing remitting MS (RE). In one the embodiment, the subject has been diagnosed as having RE MS, or is at risk of developing RE MS.

### Methods of Treatment

An aspect described herein provides methods of treating a central nervous system (CNS) disease or disorder comprising administering to a subject diagnosed with the CNS disease or disorder a composition comprising a population of genetically modified, programmed cell death-1 receptor ligand (PD-LW-expressing hematopoietic stem cells (HSCs), wherein the CNS disease or disorder involves inflammation of the CNS.

In one embodiment, the CNS disease or disorder is selected from the group consisting of MS, Systemic lupus erythematosus (SLE), inflammatory brain disease, central nervous system vasculitis, Neuromyelitis Optica Spectrum Disorder. In some embodiments, the CNS disease or disorder is a neuroinflammatory or neurodegenerative disease. In some embodiments, the CNS disease or disorder is selected from the group consisting of: Alzheimer's disease, Parkinson's disease, amyotrophic lateral sclerosis, acute disseminated encephalomyelitis, acute optic neuritis, transverse myelitis, neuromyelitis optica spectrum disorder, brain injury, autoimmune encephalitis, anti-NMDA receptor encephalitis, Rasmussen's encephalitis, acute necrotizing encephalopathy of childhood, opsoclonus-myoclonus-ataxia syndrome, and GLUT-1 deficiency syndrome.

In one embodiment, the method further comprises the step, prior to administering, of diagnosing a subject as having the CNS disease or disorder.

In one embodiment, the method further comprises the step, prior to administering, of receiving the results of an assay that diagnoses a subject as having the CNS disease or disorder.

A skilled clinician can diagnose a subject as having a CNS disease or disorder by, e.g., determining if a subject presents with any symptoms associated with a CNS disease or disorder, including, but not limited to, persistent or sudden onset of a headache, loss of feeling or tingling, weakness or loss of muscle strength, loss of sight or double vision, memory loss, impaired mental ability, or lack of coordination. Exemplary symptoms of SLE include butterfly-shaped rash on face or body, skin lesions, and joint pain. Exemplary symptoms of inflammatory brain disease include stroke and speech impairment. Exemplary symptoms of central nervous system vasculitis include significant confusion, stroke, and seizures. Exemplary symptoms of Neuromyelitis Optica Spectrum Disorder include sudden blindness and paralysis in legs or arms. Further, a CNS disease and disorders can be diagnosed by a skilled practitioner using standard assays in the field, for example, a computerized tomography (CT) scan, a lumbar puncture, e.g., to measure the pressure of the cerebrospinal fluid, magnetic resonance imaging (MRI) or magnetic resonance angiogram (MRA), Electroencephalography (EEG) to look at brain activity, or Electromyography (EMG) to test nerve and muscle function.

In one embodiment, the subject is at risk of developing a CNS disease or disorder. One aspect herein the provides a method of preventing CNS disease or disorder comprising administering to a subject at risk of developing a CNS disease or disorder a composition comprising a population of genetically modified, PD-L1⁺-expressing HSCs. In certain embodiments, the subject is identified as being at risk of developing the CNS disease or disorder prior to administration. Risk factors for a CNS disease or disorder can be identified by a skilled clinician and include, but are not limited to, a family history of the CNS disease or disorder, an infection of the CNS, diagnosis of certain other autoimmune diseases, and surgical procedure of the CNS.

In one embodiment, administering a composition described herein decreases the frequency of a symptom associated with the CNS disease or disorder by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject having a same CNS disease or disorder that is not administered a composition described herein. One skilled in the art will be capable of determining the frequency of symptoms using standard techniques, e.g., frequent medical exams, or personal symptom diary completed by the subject.

In one embodiment, administering a composition described herein inhibits or slows the progression and/or severity of a CNS disease or disorder in a subject identified as having such disease by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% as compared to an appropriate control. As used herein, an appropriate control refers to an otherwise identical subject having a same CNS disease or disorder that is not administered a composition described herein. One skilled in the art will be capable of determining the progression and/or severity of a CNS disease or disorder using standard techniques, e.g., frequent medical exams, or personal symptom diary completed by the subject.

In one embodiment, administering a composition described herein decreases the risk of a developing a CNS disease by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject having a same risk of developing the CNS disease that is not administered a composition described herein.

In one embodiment, the method further comprises the step, prior to administering, of obtaining an unmodified HSCs from bone marrow, umbilical cord, amniotic fluid, chorionic villi, cord blood, placental blood or peripheral blood, and genetically modifying the obtained HSCs by introducing an exogenous copy of a nucleic acid encoding a PD-L1.

Another aspect described herein provides method of treating a MS comprising administering to a subject diagnosed with MS a composition comprising a population of genetically modified, PD-L1⁺-expressing HSCs.

In one embodiment, the method further comprises the step, prior to administering, of diagnosing a subject as having MS.

In one embodiment, the method further comprises the step, prior to administering, of receiving the results of an assay that diagnoses a subject as having MS.

A skilled clinician can diagnose a subject as having MS by, e.g., determining if a subject presents with any symptoms associated with MS, including, but are not limited to, fatigue; tingling, pain or numbness of extremities; loss of balance, trouble walking; changes in vision; clinical depression; impaired cognitive function; poor muscle coordination; sexual dysfunction; slurred speech and stuttering; and bladder and bowel dysfunction. A skilled clinician can perform a physical examination to reveal additional MS signs, such as irregular eye movement; changes in speech; loss of coordination; sensory disturbances; changes in reflexes; and/or weakness/spasticity in arms or legs. Further, MS can be diagnosed via MRI or MRA to detect distinctive lesions or lesion scars and/or inflammation in the central nervous system (e.g., brain, spine and optic nerve) associated with MS. Additional assays, such as a spinal tap to obtain cerebrospinal fluid or blood tests, can be done to confirm an MS diagnosis.

In one embodiment, administering a composition described herein decreases the frequency of a symptom associated with MS by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject having a same classification of MS that is not administered a composition described herein. One skilled in the art will be capable of determining the frequency of symptoms using standard techniques, e.g., frequent medical exams, or personal symptom diary completed by the subject.

In one embodiment, administering a composition described herein decreases the severity of a symptom associated with MS by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject having a same classification of MS that is not administered a composition described herein. One skilled in the art will be capable of determining the severity of symptoms using standard techniques, e.g., frequent medical exams and assays for MS as described herein.

In one embodiment, administering a composition described herein decreases the quantity of symptoms associated with MS by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject having a same classification of MS that is not administered a composition described herein. One skilled in the art will be capable of determining the quantity of symptoms using standard techniques, e.g., frequent medical exams, or personal symptom diary completed by the subject.

In one embodiment, administering a composition described herein decreases the frequency and/or duration of a relapse associated with MS by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject having a same classification of MS that is not administered a composition described herein. One skilled in the art will be capable of determining the frequency and/or duration of relapse using standard techniques, e.g., frequent medical exams, or personal symptom diary completed by the subject.

In one embodiment, administering a composition described herein increases the frequency and/or duration, of a remittance period associated with MS by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 1×, 2×, 3×, 4×, 5×, 6×, 7×, 8×, 9×, 10× or more as compared to an appropriate control. As used herein, an appropriate control refers to an otherwise identical subject having a same classification of MS that is not administered a composition described herein. One skilled in the art will be capable of determining the frequency and/or duration of remittance using standard techniques, e.g., frequent medical exams, or personal symptom diary completed by the subject.

In one embodiment, administering a composition described herein delays the progression of MS by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject having a same classification of MS that is not administered a composition described herein. One skilled in the art will be capable of determining the progression using standard techniques, e.g., described herein above.

In one embodiment, the subject is at risk of developing MS. One aspect herein provides a method of preventing MS comprising administering to a subject at risk of developing MS a composition comprising a population of genetically modified, PD-L1⁺-expressing HSCs. In certain embodiments, the subject is identified as being at risk of developing MS prior to administration a composition described herein. Risk factors for MS can be identified by a skilled clinician and include, but is not limited to, age (e.g., MS usually affects people somewhere between the ages of 16 and 55); sex (e.g., females are more than two to three times as likely as males are to have relapsing-remitting MS); family history (e.g., a subject is at a higher risk of developing MS if the subject's parent or sibling has had MS); certain infections (e.g., a variety of viruses have been linked to MS, including Epstein-Barr, the virus that causes infectious mononucleosis); race (e.g., white people, particularly those of Northern European descent, are at highest risk of developing MS, whereas people of Asian, African or Native American descent have the lowest risk); Climate (e.g., MS is far more common in countries with temperate climates, including Canada, the northern United States, New Zealand, southeastern Australia and Europe); vitamin D deficiency; certain autoimmune diseases (e.g., a subject having a thyroid disease, type 1 diabetes or inflammatory bowel disease have a slightly higher risk of developing MS); and smoking.

In one embodiment, administering a composition described herein decreases the risk of a developing MS by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject having the same risk as developing MS that is not administered a composition described herein.

Another aspect described herein provides a method of treating inflammation of the CNS comprising administering to a subject diagnosed with inflammation of the CNS a composition comprising a population of genetically modified, PD-L1⁺-expressing HSCs. Inflammation of the CNS can be inflammation of the spinal cord and/or the brain. For example, inflammation can be present in any of the lobes of the brain (i.e., temporal lobe, occipital lobe, parietal lobe, and frontal lobe), any of the regions of the brain (i.e., basal ganglia, cerebellum, Broca's area, corpus callosum, medulla oblongata, hypothalamus, thalamus, and amygdala), in the brain stem, or in any of the sections of the spinal cord (i.e., cervical, thoracic, lumbar, sacrum and coccyx). Inflammation can be present in the grey or white matter of the CNS.

Inflammation of the CNS can be diagnosed by a skilled clinician using standard techniques, e.g. immunohistochemistry to measure specific markers of inflammation in cerebrospinal fluid or blood (e.g., microglial activation (Iba1), astrocytic response (GFAP), and neuronal loss (NeuN or Fluorojade for dying neurons)), or non-invasive imaging, such as MRI, CT scan, or positron emission tomography (PET) scan in combination with specific radiotracers, e.g., translocator protein-18 kDa (TSPO).

In one embodiment, administering a composition described herein reduced or decreases inflammation of the CNS by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject having a same level of inflammation that is not administered a composition described herein. One skilled in the art can measure the level of inflammation in the CNS using assays described herein above.

In one embodiment, administering a composition described herein decreases the number of neutrophils in the CNS by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject that is not administered a composition described herein. Neutrophils can be identified by one skilled in the art by, e.g., FACS sorting for known neutrophil markers, e.g., CEACAM-8, CD11b/Integrin alpha M, and CD33.

In one embodiment, administering a composition described herein increases the number of CD3+ T cells in the CNS by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or 1×, 2×, 3×, 4×, 6×, 7×, 8×, 9×, 10×, or more as compared to an appropriate control. As used herein, an appropriate control refers to an otherwise identical subject that is not administered a composition described herein. CD3+ T can be identified by one skilled in the art by, e.g., FACS sorting for known neutrophil markers, e.g., CD3.

In one embodiment, the subject is at risk of developing inflammation of the CNS. One aspect herein the provides a method of preventing inflammation of the CNS comprising administering to a subject at risk of developing inflammation of the CNS a composition comprising a population of genetically modified, PD-L1⁺-expressing HSCs. In certain embodiments, the subject is identified as being at risk of developing inflammation of the CNS prior to administration. Risk factors for inflammation of the CNS can be identified by a skilled clinician and include, but are not limited to, a bacterial or viral infection of the CNS, a CNS disease or disorder, or an injury to the CNS, such as a traumatic brain injury, stroke, concussion, or the like.

In one embodiment, administering a composition described herein decreases the risk of a developing inflammation by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to an otherwise identical subject having a same risk of developing inflammation, e.g., exhibits a similar risk factor, that is not administered a composition described herein.

Another aspect provided herein is a method of treating a CNS disease or disorder associated with inflammation of the CNS in a subject comprising (a) providing a population of unmodified hematopoietic stem cells (HSCs); (b) contacting the population of unmodified HSCs of (a) with a vector carrying an exogenous copy of a nucleic acid encoding a PD-L1, thereby obtaining modified HSCs; (c) ex vivo culturing the resultant modified HSCs from the contacting; (d) establishing expression of PD-L1 on the modified HSCs, thereby producing a population of PD-L1⁺-expressing HSCs; and (e) transplanting said population of PD-L1⁺-expressing HSCs into a recipient subject having a CNS disease or disorder associated with inflammation of the CNS, thereby treating the CNS disease or disorder in the recipient subject.

Another aspect provided herein is a method of treating MS in a subject comprising (a) providing a population of unmodified hematopoietic stem cells (HSCs); (b) contacting the population of unmodified HSCs of (a) with a vector carrying an exogenous copy of a nucleic acid encoding a PD-L1, thereby obtaining modified HSCs; (c) ex vivo culturing the resultant modified HSCs from the contacting; (d) establishing the expression of PD-L1 on the modified HSCs, thereby producing a population of PD-L1⁺-expressing HSCs; and (e) transplanting said population of PD-L1⁺-expressing HSCs into a recipient subject having MS, thereby treating MS in the recipient subject.

Another aspect provided herein is a method of treating or preventing inflammation of the CNS in a subject comprising (a) providing a population of unmodified hematopoietic stem cells (HSCs); (b) contacting the population of unmodified HSCs of (a) with a vector carrying an exogenous copy of a nucleic acid encoding a PD-L1, thereby obtaining modified HSCs; (c) ex vivo culturing the resultant modified HSCs from the contacting; (d) establishing expression of PD-L1 on the modified HSCs, thereby producing a population of PD-L1⁺-expressing HSCs; and (e) transplanting said population of PD-L1⁺-expressing HSCs into a recipient subject having or at risk of having inflammation of the CNS, thereby preventing or treating inflammation of the CNS in the recipient subject.

Another aspect provided herein is a method of treating a CNS disease or disorder associated with inflammation of the CNS in a subject, the method comprising (a) receiving a population of PD-L1⁺-expressing HSCs; and (b) transplanting said population of PD-L1⁺-expressing HSCs into a recipient subject having a CNS disease or disorder associated with inflammation of the CNS, thereby treating the CNS disease or disorder in the recipient subject.

Another aspect provided herein is a method of treating MS in a subject comprising (a) receiving a population of PD-L1⁺-expressing HSCs; and (b) transplanting said population of PD-L1⁺-expressing HSCs into a recipient subject having MS, thereby treating MS in the recipient subject.

Another aspect provided herein is a method of treating or preventing inflammation of the CNS in a subject comprising (a) receiving a population of PD-L1⁺-expressing HSCs; and (b) transplanting said population of PD-L1⁺-expressing HSCs into a recipient subject having or at risk of having inflammation of the CNS, thereby preventing or treating inflammation of the CNS in the recipient subject.

The population of unmodified or modified HSCs can be autologous to the recipient subject, allogeneic to the recipient subject, or xenogeneic to the recipient subject.

In some embodiments, the administered hematopoietic stem cell, or population thereof, differentiates into a blood cell following administration to subject. In some embodiments, the HSC is committed to the blood lineage following transplantation into a subject. Differentiation of HSCs to fully differentiated blood cells is believed to be an irreversible process under normal physiological conditions. Hematopoietic lineage specification takes place within the bounds of strict lineal relationships: for example, megakaryocyte progenitors give rise to megakaryocytes and ultimately platelets, but not to any other blood lineages. A HSC can differentiate into all blood cell types. Non-limiting examples of blood cells that a HSC can differentiate into include a myeloid progenitor, a lymphoid progenitor, a megakaroblast, a promegakarocyte, a megakaryocyte, a thrombocyte, a proerythroblast, a basophilic erythroblast, a polychromatic erythroblast, a orthochromatic erythroblast, a polychromatic erythrocyte, an erythrocyte, a myeloblast, a B. promyelocyte, a B. myelocyte, a B. metamyelocyte, a B. band, a Basophil, a N. promyelocyte, a N. myelocyte, a N. metamyelocyte, a N. band, a neutrophil, an E. promyelocyte, an E. myelocyte, an E. metamyelocyte, an E. band, an eosinophil, a monoblast, a promonocyte, a monocyte, a macrophage, a myeloid dendritic cell, a lymphoblast, a prolymphocyte, a small lymphocyte, a B lymphocyte, a T lymphocyte, a plasma cell, a large granular lymphocyte, and a lymphoid dendritic cell.

### PD-L1

Programmed cell death protein 1, also known as PD-1 and cluster of differentiation 279 (CD279), is a receptor protein that in humans is encoded by the PDCD1 gene. PD-1 is a cell surface receptor that belongs to the immunoglobulin superfamily and is expressed on activated T cells and pro-B cells. PD-1 binds two ligands, PD-L1 (also known as B7 homolog 1 (B7-H1) or cluster of differentiation 274 (CD 274)) and PD-L2. The two ligands of PD-1, PD-L1 and PD-L2, are members of the B7 family. PD-1 and its ligands play an important role in down regulating the immune system by preventing the activation of T-cells. PD-L1/PD-1 interaction deactivates T cell's cytotoxic activity and leads to the inhibition of the adaptive immune system. This in turn reduces autoimmunity and promotes self-tolerance. The inhibitory effect of PD-1 is accomplished through a dual mechanism of promoting apoptosis (programmed cell death) in antigen specific T-cells in lymph nodes while simultaneously reducing apoptosis in regulatory T cells (suppressor T cells).

PD-L1 plays a major role in suppressing the immune system during particular events such as pregnancy, tissue allografts, autoimmune disease and other disease states such as hepatitis. Normally the immune system reacts to foreign antigens where there is some accumulation in the lymph nodes or spleen which triggers a proliferation of antigen-specific CD8+ T cell. The formation of PD-1 receptor/PD-L1 or B7.1 receptor/PD-L1 ligand complex transmits an inhibitory signal which reduces the proliferation of these CD8+ T cells at the lymph nodes and supplementary to that PD-1 is also able to control the accumulation of foreign antigen specific T cells in the lymph nodes through apoptosis which is further mediated by a lower regulation of the gene BCL-2.

As used herein, “PD-L1” refers to one of the ligands of the receptor PD-1, a 40 kDa type 1 transmembrane protein encoded by the CD274 gene (Gene ID: 29126). Other abbreviated symbols for PD-L1 are B7-H, B7H1, PDCD1L1, PDCD1LG1, and PDL1PD-L1. The human CD274 gene can be found on chromosome 9 at the location NC 000009.12 (5450381 . . . 5470567) according to the Assembly from the Genome Reference Consortium Human Build 38 patch release 2 (GRCh38.p2), under RefSeq or GENBANK assembly accession No: GCF_000001405.28, dated Dec. 5, 2014. The mRNA of the human PD-L1 can be found at GENBANK accession Nos: NM 001267706.1, NM 014143.3, BC113734.1, BC113736.1, BC074984.2 and BC069381.1. PD-L1 can refer to human PD-L1, including naturally occurring variants, molecules, and alleles thereof. In other embodiments, PD-L1 refers to the mammalian PD-L1 of, e.g., mouse, rat, rabbit, dog, cat, cow, horse, pig, and the like.

In one embodiment, a cDNA of the human PD-L1 isoform b precursor (variant 2) has the DNA sequence of SEQ ID NO: 1.

(SEQ ID NO: 1)
at
gaggatattt gctgtcttta tattcatgac ctactggcat
ttgctgaacg ccccatacaa caaaatcaac caaagaattt
tggttgtgga tocagtcacc tctgaacatg aactgacatg
tcaggctgag ggctacccca aggccgaagt catctggaca
agcagtgacc atcaagtcct gagtggtaag accaccacca
ccaattccaa gagagaggag aagcttttca atgtgaccag
cacactgaga atcaacacaa caactaatga gattttctac
tgcactttta ggagattaga tcctgaggaa aaccatacag
ctgaattggt catcccagaa ctacctctgg cacatcctcc
aaatgaaagg actcacttgg taattctggg agccatctta
ttatgccttg gtgtagcact gacattcatc ttccgtttaa
gaaaagggag aatgatggat gtgaaaaaat gtggcatcca
agatacaaac tcaaagaagc aaagtgatac acatttggag
gagacgtaa.

This variant 2 (SEQ ID NO: 1) represents the shorter transcript and encodes the shorter isoform b.

In one embodiment, a cDNA of the human PD-L1 isoform a precursor (variant 1) has the DNA sequence of SEQ ID NO: 2.

(SEQ ID NO: 2)
at
gaggatattt gctgtcttta tattcatgac ctactggcat
ttgctgaacg catttactgt cacggttccc aaggacctat
atgtggtaga gtatggtagc aatatgacaa ttgaatgcaa
attcccagta gaaaaacaat tagacctggc tgcactaatt
gtctattggg aaatggagga taagaacatt attcaatttg
tgcatggaga ggaagacctg aaggttcagc atagtagcta
cagacagagg gcccggctgt tgaaggacca gctctccctg
ggaaatgctg cacttcagat cacagatgtg aaattgcagg
atgcaggggt gtaccgctgc atgatcagct atggtggtgc
cgactacaag cgaattactg tgaaagtcaa tgccccatac
aacaaaatca accaaagaat tttggttgtg gatccagtca
cctctgaaca tgaactgaca tgtcaggctg agggctaccc
caaggccgaa gtcatctgga caagcagtga ccatcaagtc
ctgagtggta agaccaccac caccaattcc aagagagagg
agaagctttt caatgtgacc agcacactga gaatcaacac
aacaactaat gagattttct actgcacttt taggagatta
gatcctgagg aaaaccatac agctgaattg gtcatcccag
aactacctct ggcacatcct ccaaatgaaa ggactcactt
ggtaattctg ggagccatct tattatgcct tggtgtagca
ctgacattca tcttccgttt aagaaaaggg agaatgatgg
atgtgaaaaa atgtggcatc caagatacaa actcaaagaa
gcaaagtgat acacatttgg aggagacgta a.

This variant 1 (SEQ ID NO: 2) represents the longest transcript and encodes the longer isoform a.

In one embodiment, the nucleic acid encoding PD-L1 encodes a human PD-L1 polynucleotide.

In one embodiment, the nucleic acid encodes the human PD-L1 polynucleotide having the amino acid sequence of human PD-L1 isoform b precursor (variant 2) according to SEQ ID NO: 3.

(SEQ ID NO: 3)
MRIFAVFIFM TYWHLLNAPY NKINQRILVV
DPVTSEHELT CQAEGYPKAE VIWTSSDHQV
LSGKTTTINS KREEKLENVT STLRINTTIN
EIFYCTERRL DPEENHTAEL VIPELPLAHP
PNERTHLVIL GAILLCLGVA LTFIFRLRKG
RMMDVKKCGI QDTNSKKQSD THLEET

In one embodiment, the nucleic acid encodes the human PD-L1 polynucleotide having the amino acid sequence of human PD-L1 isoform a precursor (variant 1) according to SEQ ID NO: 4.

(SEQ ID NO: 4)
MRIFAVFIFM TYWHLLNAFT VTVPKDLYVV
EYGSNMTIEC KFPVEKQLDL AALIVYWEME
DKNIIQFVHG EEDLKVQHSS YRORARLLKD
QLSLGNAALQ ITDVKLQDAG VYRCMISYGG
ADYKRITVKV NAPYNKINQR ILVVDPVTSE
HELTCQAEGY PKAEVIWTSS DHQVLSGKTT
TTNSKREEKL ENVTSTLRIN TTTNEIFYCT
FRRLDPEENH TAELVIPELP LAHPPNERTH
LVILGAILLC LGVALTFIFR LRKGRMMDVK
KCGIQDTNSK KQSDTHLEET

In one embodiment of the composition of modified HSCs described herein, the nucleic acid encoding PD-L1 is a complementary DNA (cDNA). In one embodiment, the cDNA encoding PD-L1 is an mRNA. In one embodiment, the mRNA is SEQ ID NO: 1 or 2. In other embodiments, the mRNA is derived from the GenBank accession Nos: NM_001267706.1, NM_014143.3, BC113734.1, BC113736.1, BC074984.2 or BC069381.1.

In another embodiment of the composition of modified HSCs described herein, the nucleic acid encoding PD-L1 is a genomic DNA. In one embodiment, the genomic DNA encoding PD-L1 is derived from the GenBank assembly accession No: GCF_000001405.28.

### Hematopoietic Stem Cells

HSCs are known to give rise to committed hematopoietic progenitor cells (HPCs) that are capable of generating the entire repertoire of mature blood cells over the lifetime of an organism. The term “hematopoietic stem cell” or “HSC” generally refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,163; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827; the contents of which are incorporated herein by reference in their entireties). When transplanted into lethally irradiated animals or humans, hematopoietic stem and progenitor cells can repopulate the erythroid, neutrophil-macrophage, megakaryocyte and lymphoid hematopoietic cell pool.

In one embodiment, the term “hematopoietic stem cell” or “HSC” refers to a stem cell that have the following cell surface markers: CD34⁺, CD59⁺, Thy1/CD90⁺, CD38^{lo/-}, and C-kit/CD117⁺. In one embodiment, the term “hematopoietic stem cell” or “HSC” refers to a stem cell that is at least CD34⁺. In one embodiment, the term “hematopoietic stem cell” or “HSC” refers to a stem cell that is at least CD38lo/⁻. In one embodiment, the term “hematopoietic stem cell” or “HSC” refers to a stem cell that is at least CD34⁺ and CD38^{lo/-}. In one embodiment, the term “hematopoietic stem cell” or “HSC” refers to a stem cell that is at least lin⁻. In one embodiment, the term “hematopoietic stem cell” or “HSC” refers to a stem cell that is at least CD34⁺, and lin⁻. In one embodiment, the term “hematopoietic stem cell” or “HSC” refers to a stem cell that is at least CD34⁺, CD38^{lo/-} and lin⁻. In one embodiment, the term “hematopoietic stem cell” or “HSC” refers to a stem cell that is at least CD34⁺ and C-kit/CD117⁺. In one embodiment, the term “hematopoietic stem cell” or “HSC” refers to a stem cell that is at least CD34⁺, CD38^{lo/-} and C-kit/CD117⁺. In another embodiment, as used herein, the term “hematopoietic stem cell” or “HSC” includes hematopoietic stem and progenitor cells (HSPC).

Mature blood cells have a finite lifespan and must be continuously replaced throughout life. Blood cells are produced by the proliferation and differentiation of a very small population of pluripotent HSCs that also have the ability to replenish themselves by self-renewal. HSCs are multipotent, self-renewing progenitor cells that develop from mesodermal hemangioblast cells. HSCs are the blood cells that give rise to all the other blood cells, that includes all the differentiated blood cells from the erythroid, lymphoid and myeloid lineages. HSCs are located in the adult bone marrow, peripheral blood, and umbilical cord blood.

During differentiation, the progeny of HSCs progress through various intermediate maturational stages, generating multi-potential hematopoietic progenitor cells and lineage-committed hematopoietic progenitor cells, prior to reaching maturity. Bone marrow (BM) is the major site of hematopoiesis in humans and, under normal conditions, only small numbers of HSCs and hematopoietic progenitor cells can be found in the peripheral blood (PB). Treatment with cytokines (in particular granulocyte colony-stimulating factor; G-CSF), myelosuppressive drugs used in cancer treatment, and compounds that disrupt the interaction between hematopoietic cells and BM stromal cells can rapidly mobilize large numbers of stem and progenitor cells into the circulation.

The HSCs, similar to the hematopoietic progenitor cells, are capable of proliferation and giving rise to more progenitor cells having the ability to generate a large number of mother cells that can in turn give rise to differentiated or differentiable daughter cells. The daughter cells themselves can be stimulated to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate.

Peripheral blood progenitor cells (PBPC) have become the preferred source of hematopoietic progenitor cells and HSCs for allogeneic and autologous transplantation because of technical ease of collection and shorter time required for engraftment. Traditionally, granulocyte-colony stimulating factor (G-CSF) has been used to stimulate more PBPC and release of hematopoietic progenitor cells from the bone marrow. Although regimens using G-CSF usually succeed in collecting adequate numbers of PBPC from healthy donors, 5%-10% will mobilize stem cells poorly and may require multiple large volume apheresis or bone marrow harvesting.

In one embodiment, unmodified HSCs are obtained or isolated from bone marrow, umbilical cord, chorionic villi, amniotic fluid, placental blood, cord blood or peripheral blood. One skilled in the art will know how to obtain or isolate such cell population using standard protocol known in the art and/or described herein. Methods of mobilizing HSCs from the places of origin or storage are known in the art. For example, treatment with cytokines, in particular granulocyte colony-stimulating factor (G-CSF) and compounds (e.g., plerixafor, a chemokine CXCR4 antagonist) that disrupt the interaction between HSCs and bone marrow (BM) stromal cells can rapidly mobilize large numbers of hematopoietic stem and hematopoietic progenitor cells into the circulation. In one embodiment, CD34″ HSCs are obtained or isolated from the bone marrow, umbilical cord, chorionic villi, amniotic fluid, placental blood, cord blood or peripheral blood.

In one embodiment, the HSCs are derived from a healthy individual, e.g., a subject not having a disease or disorder. Alternatively, the HSCs are derived from an individual with a diagnosed disease or disorder, e.g., a CNS disease or disorder, such as MS. In one embodiment, the HSCs are derived from the subject which that will be administered to, i.e., the HSCs are autologous to the subject.

In various embodiments, the population of PD-L⁺-expressing HSCs are autologous, allogeneic, or xenogeneic to the subject.

### Modified PD-L1-Expressing HSCs

Methods described herein require the administration of modified, PD-L1⁺-expressing HSCs to a subject. In one embodiment, the modified PD-L1⁺-expressing HSCs carry an exogenous copy of a nucleic acid encoding a programmed cell death-1 receptor ligand (PD-L1). It is preferred that the nucleic acid is integrated into the genome of the cells. Alternatively, the HSCs can transiently express an exogenous copy of a nucleic acid encoding PD-L1. The nucleic acid can be complementary DNA (cDNA), or alternatively can be genomic DNA.

In one embodiment, the cDNA comprises, consists of, or consists essentially of SEQ ID NO: 1 or 2.

The nucleic acid can be introduced into the cell using various known methods, for example, as naked DNA or included in a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, that is, unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., a PD-L1) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

In some embodiments, the viral vector is a lentiviral vector comprising partial (e.g., split) gene lentiviral sequences and/or non-lentiviral sequences (e.g., sequences from other retroviruses). The viral vector can include, but is not limited to, promoter, packaging signal, LTR(s), polypurine tracts, and a reverse response element (RRE).

In some embodiments, the lentiviral vector comprises a viral LTR promoter. In some embodiments, the lentiviral vector comprises a heterologous promoter replacing the viral LTR promoter. Examples of heterologous promoters which can be used include, but are not limited to, a spleen focus-forming virus (SFFV) promoter, a tetracycline-inducible (TET) promoter, a β-globin locus control region and a β-globin promoter (LCR), and a cytomegalovirus (CMV) promoter. In some embodiments, the promoter is a regulatable promoter, an inducible promoter, for the regulating the production of PD-L1. For example, a Tetracyclin-inducible or Doxycyclin-inducible promoter can be used.

The promoter of the lentiviral vector can be one which is naturally (i.e., as it occurs with a cell in vivo) or non-naturally associated with the 5′ flanking region of a particular gene. Promoters can be derived from eukaryotic genomes, viral genomes, or synthetic sequences. Promoters can be selected to be non-specific (active in all tissues) (e.g., SFFV), tissue specific (e.g., (LCR), regulated by natural regulatory processes, regulated by exogenously applied drugs (e.g., TET), or regulated by specific physiological states such as those promoters which are activated during an acute phase response or those which are activated only in replicating cells. Non-limiting examples of promoters in the present disclosure include the spleen focus-forming virus promoter, a tetracycline-inducible promoter, a β-globin locus control region and a β-globin promoter (LCR), a cytomegalovirus (CMV) promoter, retroviral LTR promoter, cytomegalovirus immediate early promoter, SV40 promoter, and dihydrofolate reductase promoter. The promoter can also be selected from those shown to specifically express in the select cell types such as HSCs and their progenies. In one embodiment, the promoter of the vector is cell specific such that gene expression is restricted to red blood cells. Erythrocyte-specific expression is achieved by using the human β-globin promoter region and locus control region (LCR).

In one embodiment, full-length cDNA encoding murine PD-L1 was cloned into the transfer plasmid pHAGE-fullEF1a-TRE-IZsGreen, containing internal EF1a promoter, IRES-ZsGreen sequence and Tet-responsive element. In one embodiment, mPDL1 and ZsGreen expression is induced in the presence of doxycycline. In one embodiment, the expression of the bicistronic transcript is driven by the human elongation factor 1 alpha (EF1α) promoter.

In one embodiment, lentivirus vectors were obtained by co-transfection into 293T cells of the murine PD-L1 transfer plasmid together with the packaging expression plasmids (Gag/Pol, Tat, Rev) and a plasmid encoding for VSV-G pseudotyped glycoprotein.

In one embodiment, the modified, PD-L1⁺-expressing HSCs are produced by a method comprising: (a) contacting a population of unmodified HSCs with a vector carrying an exogenous copy of a nucleic acid encoding a PD-L1, thereby obtaining modified HSCs; (b) ex vivo culturing the resultant modified cells from the contacting; and (c) establishing expression of PD-L1 on the modified HSCs, thereby producing a population of modified HSCs cells expressing PD-L1.

Also provided herein an ex vivo method of producing a population of PD-L1⁺-expressing hematopoietic stem cells (HSCs), the method comprising: (a) obtaining a population of unmodified HSCs from a subject diagnosed with a CNS disease or disorder; (b) contacting the population of unmodified HSCs with a vector carrying an exogenous copy of a nucleic acid encoding PD-L1, thereby obtaining modified HSCs; (c) ex vivo culturing the resultant modified HSCs; and (d) establishing expression of PD-L1 on the modified HSCs, thereby producing a population of modified HSCs expressing PD-L1.

In some embodiments, a population of unmodified HSCs is contacted with at least 10³ vectors or viral vectors or particles per 10⁶ HSC cells in the ex vivo transfection or transduction procedure. The vector carries an exogenous copy of a nucleic acid encoding a PD-L1. Other vector dosage ranges set forth herein for contacting with the sample of HSCs is exemplary only and are not intended to limit the scope or practice of the claimed composition or methods described herein. In one embodiment, the vector dosage is ranges from 10³-10⁸ viral particles/10⁶ HSC cells. In other embodiments, the vector dosage is ranges from 10³-10⁵ viral particles/10⁶ HSC cells, 10⁴-10⁶ viral particles/10⁶ HSC cells, 10⁵-10⁷ viral particles/10⁶ HSC cells, 10³-10⁸ viral particles/10⁶ HSC cells. In one embodiment, the dosage is about 10⁴ viral particles/10⁶ HSC cells.

In one embodiment, establishing the expression of PD-L1 on the modified HSCs is determining that the population of modified HSCs have an at least 1-fold increase in the number of PD-L1⁺-expressing HSCs compared to a population of non-modified HSCs. In one embodiment, establishing the expression of PD-L1 on the modified HSCs is determining that the population of modified HSCs have an at least a 2-fold, 3-fold, 4-fold, 5-fold, 6-fold, 7-fold, 8-fold, 9-fold, 10-fold or more increase in the number of PD-L1⁺-expressing HSCs compared to a population of non-modified HSCs. In one embodiment, establishing the expression of PD-L1 on the modified HSCs is determining that the population of modified HSCs have physiologically relevant levels of PD-L1.

In one embodiment, the population of modified HSCs have physiologically relevant levels of PD-L1.

In some embodiments, the method further comprises the step of sorting, isolating or enriching PD-L1⁺-expressing HSCs in a population of HSCs. In some embodiments, more than 60%, 65% 70%, 75%, 80%, 85%, 90%, 95%, or 99% or more of the sorted, isolated, or enriched population of HSCs are genetically modified PD-L1⁺-expressing HSCs.

In some embodiments, a population of modified HSCs expressing PD-L1 have at least a 2-fold, 3-fold, 3-fold, 4-fold, 5-fold, 6-fold, 7-fold, 8-fold, 9-fold, 10-fold or more increase in the PD-L1 expression level compared to a population of non-modified HSCs. In some embodiments, a population of modified HSCs expressing PD-L1 have about a 2-fold, 3-fold, 3-fold, 4-fold, 5-fold, 6-fold, 7-fold, 8-fold, 9-fold, 10-fold or more increase in the PD-L1 expression level compared to a population of non-modified HSCs. In some embodiments, a population of modified HSCs expressing PD-L1 have between 1 and 2-fold, between 2 and 3-fold, between 3 and 4-fold, between 4 and 5-fold, between 5 and 6-fold, between 6 and 7-fold, between 7 and 8-fold, between 8 and 9-fold, or between 9 and 10-fold increase in the PD-L1 expression level compared to a population of non-modified HSCs.

In one embodiment, the modified, PD-L1⁺-expressing HSCs are mammalian, e.g., human.

In one embodiment, the modified, PD-L1⁺-expressing HSCs are obtained by genetically modifying HSCs obtained from bone marrow, umbilical cord, amniotic fluid, chorionic villi, cord blood, placental blood or peripheral blood.

In one embodiment, the modified, PD-L1⁺-expressing HSCs are obtained by genetically modifying HSCs obtained from mobilized peripheral blood.

In one embodiment, the modified, PD-L1⁺-expressing HSCs ex vivo cultured before, or after, or both before and after the introduction of the exogenous copy of a nucleic acid encoding a PD-L1.

In one embodiment, the modified, PD-L1-expressing HSCs has at least one, e.g., at least 2, 3, 4, or 5) of the cell surface marker characteristic of HSCs: CD34⁺, CD59⁺, Thy1/CD90⁺, CD38^{lo/-} and C-kit/CD117⁺. Preferably, the HSCs have at least two of these markers. In one embodiment, the modified, PD-L1-expressing HSCs are CD34⁺, CD59⁺, Thy1/CD90⁺, CD38^{lo/-}, and C-kit/CD117⁺. In one embodiment, the modified, PD-L1-expressing HSCs are CD133⁺. In one embodiment, the modified, PD-L1-expressing HSCs are CD34⁺ cells, CD38^{lo/-} cells, or c-kit⁺ cells. HSCs can be selected for the presence of a desired surface marker, e.g., CD34⁺, CD38^{lo/-}, c-kit⁺, prior to the contacting the HSC with the vector carrying the exogenous copy of the nucleic acid described herein. Positive or negative selection for the described surface markers can be performed by a skilled person using any method known in the art, e.g., using an antibody-specific immunomagnetic bead or FACS sorting.

In one embodiment, the modified, PD-L1-expressing HSCs are hematopoietic progenitor cells. Hematopoietic progenitor cells can be, e.g., CD34⁺ cells, CD38^{lo/-} cells, CD133⁺, or c-kit⁺ cells. Hematopoietic progenitor cells can be selected for the presence of a desired surface marker, e.g., CD34⁺, CD38^{lo/-}, CD133⁺, c-kit⁺, prior to the contacting the hematopoietic progenitor cells with the vector carrying the exogenous copy of the nucleic acid described herein. Positive or negative selection for the described surface markers can be performed by any method known in the art, e.g., using an antibody-specific immunomagnetic bead or FACS sorting.

In one embodiment, the modified, PD-L1-expressing HSCs are erythroid progenitor cells. Erythroid progenitor cells can be, e.g., CD34⁺ cells. Erythroid progenitor cells can be selected for the presence of a desired surface marker, CD34⁺, prior to the contacting the erythroid progenitor cells with the vector carrying the exogenous copy of the nucleic acid described herein. Positive selection for the described surface markers can be performed by any method known in the art, e.g., using an anti-CD34 immunomagnetic bead or FACS sorting.

In one embodiment, the modified, PD-L1-expressing HSCs are erythroid cells. Erythroid cells can be, e.g., CD34⁺ cells. Erythroid cells can be selected for the presence of a desired surface marker, CD34⁺, prior to the contacting the erythroid cells with the vector carrying the exogenous copy of the nucleic acid described herein. Positive selection for the described surface markers can be performed by any method known in the art, e.g., using an anti-CD34 immunomagnetic bead or FACS sorting.

In one embodiment, the modified, PD-L1-expressing HSCs or hematopoietic progenitor cells described herein have the cell surface marker CD71 and Ter119, which are characteristic of the erythroid lineage.

In one embodiment, a cell described herein is positively or negatively selected for a particular surface marker described herein (e.g., HSCs are positively or negatively selected for CD59⁺, Thy1/CD90⁺, CD38^{lo/-}, CD133⁺ and C-kit/CD117⁺) prior to contacting with a vector carrying the exogenous copy of the nucleic acid described herein.

Modified or unmodified HSCs described herein can be cryopreserved by any methods known in the art. Cryopreservation of the HSCs can take place any time after harvesting from a donor subject, after culture expansion following harvesting from a donor subject, and after transduction with the vector described herein. In one embodiment, the HSCs are cryopreserved after being genetically modified to express PD-L1.

As used herein, “cryopreserving” refers to the preservation of cells by cooling to low sub-zero temperatures, such as (typically) 77 K or −196° C. (the boiling point of liquid nitrogen). Cryopreservation also refers to preserving cells at a temperature between 4-10° C. At these low temperatures, any biological activity, including the biochemical reactions that would lead to cell death, is effectively stopped. Cryoprotective agents are often used at sub-zero temperatures to prevent the cells being preserved from damage due to freezing at low temperatures or warming to room temperature.

Freezing is destructive to most living cells. Upon cooling, as the external medium freezes, cells equilibrate by losing water, thus increasing intracellular solute concentration. Below about 10°−15° C., intracellular freezing will occur. Both intracellular freezing and solution effects are responsible for cell injury (Mazur, P., 1970, Science 168:939-949). It has been proposed that freezing destruction from extracellular ice is essentially a plasma membrane injury resulting from osmotic dehydration of the cell (Meryman, H. T., et al., 1977, Cryobiology 14:287-302).

Cryoprotective agents and optimal cooling rates can protect against cell injury. Cryoprotective agents which can be used include but are not limited to dimethyl sulfoxide (DMSO) (Lovelock, J. E. and Bishop, M. W. H., 1959, Nature 183:1394-1395; Ashwood-Smith, M. J., 1961, Nature 190:1204-1205), glycerol, polyvinylpyrrolidine (Rinfret, A. P., 1960, Ann. N.Y. Acad. Sci. 85:576), Dextran, trehalose, CryoSoFree (Signa Aldrich Co.) and polyethylene glycol (Sloviter, H. A. and Ravdin, R. G., 1962, Nature 196:548). The preferred cooling rate is 1° to 3° C./minute. After at least two hours, the T-cells have reached a temperature of −80° C. and can be placed directly into liquid nitrogen (−196° C.) for permanent storage such as in a long-term cryogenic storage vessel.

One aspect herein provides a population of PD-L1⁺-expressing HSCs, comprising HSCs obtained from a subject diagnosed with a CNS disease or disorder and an exogenous copy of a nucleic acid encoding PD-L1.

Another aspect herein provides a population of PD-L1⁺-expressing HSCs, comprising HSCs obtained from a subject diagnosed with MS and an exogenous copy of a nucleic acid encoding PD-L1.

Yet another aspect herein provides a population of PD-L1⁺-expressing HSCs, comprising HSCs obtained from a subject diagnosed with inflammation of the CNS and an exogenous copy of a nucleic acid encoding PD-L1.

In one embodiment, the exogenous copy of the nucleic acid encoding PD-L1 is integrated into a genome of the HSCs.

Additionally, provided herein is a pharmaceutical composition comprising any of the populations of PD-L1⁺-expressing HSCs described herein in a physiologically acceptable excipient.

The pharmaceutical composition can comprise about 1×10⁶ cells to about 3×10⁶ cells; about 1.0×10⁶ cells to about 5×10⁶ cells; about 1.0×10⁶ cells to about 10×10⁶ cells, about 10×10⁶ cells to about 20×10⁶ cells, about 10×10⁶ cells to about 30×10⁶ cells, or about 20×10⁶ cells to about 30×10⁶ PD-L1 expressing cells or HSCs or their progeny. In some embodiments, the pharmaceutical composition comprises about 1×10⁶ cells to about 30×10⁶ cells; about 1.0×10⁶ cells to about 20×10⁶ cells; about 1.0×10⁶ cells to about 10×10⁶ cells, about 2.0×10⁶ cells to about 30×10⁶ cells, about 2.0×10⁶ cells to about 20×10⁶ cells, or about 2.0×10⁶ cells to about 10×10⁶ PD-L1 expressing cells or HSCs or their progeny. In some embodiments, the pharmaceutical composition comprises about 1×10⁶ hematopoietic stem or progenitor cells, about 2×10⁶ cells, about 5×10⁶ cells, about 7×10⁶ cells, about 10×10⁶ cells, about 15×10⁶ cells, about 17×10⁶ cells, about 20×10⁶ cells about 25×10⁶ cells, or about 30×10⁶ PD-L1 expressing cells or HSCs or their progeny.

In one embodiment, the pharmaceutical composition comprises a dose ranging between 1 and 10 million cells/kg, between 1 and 2 million cells/kg, between 1 and 3 million cells/kg, between 1 and 4 million cells/kg, between 1 and 5 million cells/kg, between 1 and 6 million cells/kg, between 1 and 7 million cells/kg, between 1 and 8 million cells/kg, between 1 and 9 million cells/kg, between 2 and 3 million cells/kg, between 2 and 4 million cells/kg, between 2 and 5 million cells/kg, between 2 and 6 million cells/kg, between 2 and 7 million cells/kg, between 2 and 8 million cells/kg, between 2 and 9 million cells/kg, between 2 and 10 million cells/kg, between 3 and 4 million cells/kg, between 3 and 5 million cells/kg, between 3 and 6 million cells/kg, between 3 and 7 million cells/kg, between 3 and 8 million cells/kg, between 3 and 9 million cells/kg, between 3 and 10 million cells/kg, between 4 and 5 million cells/kg, between 4 and 6 million cells/kg, between 4 and 7 million cells/kg, between 4 and 8 million cells/kg, between 4 and 9 million cells/kg, between 4 and 10 million cells/kg, between 5 and 6 million cells/kg, between 5 and 7 million cells/kg, between 5 and 8 million cells/kg, between 5 and 9 million cells/kg, between 5 and 10 million cells/kg, between 6 and 7 million cells/kg, between 6 and 8 million cells/kg, between 6 and 9 million cells/kg, between 6 and 10 million cells/kg, between 7 and 8 million cells/kg, between 7 and 9 million cells/kg, between 7 and 10 million cells/kg, between 8 and 9 million cells/kg, between 8 and 10 million cells/kg, or between 9 and 10 million cells/kg.

In one embodiment, the dose range is between 1 and 10 million cells/kg, between 1 and 2 million cells/kg, between 1 and 3 million cells/kg, between 1 and 4 million cells/kg, between 1 and 5 million cells/kg, between 1 and 6 million cells/kg, between 1 and 7 million cells/kg, between 1 and 8 million cells/kg, between 1 and 9 million cells/kg, between 2 and 3 million cells/kg, between 2 and 4 million cells/kg, between 2 and 5 million cells/kg, between 2 and 6 million cells/kg, between 2 and 7 million cells/kg, between 2 and 8 million cells/kg, between 2 and 9 million cells/kg, between 2 and 10 million cells/kg, between 3 and 4 million cells/kg, between 3 and 5 million cells/kg, between 3 and 6 million cells/kg, between 3 and 7 million cells/kg, between 3 and 8 million cells/kg, between 3 and 9 million cells/kg, between 3 and 10 million cells/kg, between 4 and 5 million cells/kg, between 4 and 6 million cells/kg, between 4 and 7 million cells/kg, between 4 and 8 million cells/kg, between 4 and 9 million cells/kg, between 4 and 10 million cells/kg, between 5 and 6 million cells/kg, between 5 and 7 million cells/kg, between 5 and 8 million cells/kg, between 5 and 9 million cells/kg, between 5 and 10 million cells/kg, between 6 and 7 million cells/kg, between 6 and 8 million cells/kg, between 6 and 9 million cells/kg, between 6 and 10 million cells/kg, between 7 and 8 million cells/kg, between 7 and 9 million cells/kg, between 7 and 10 million cells/kg, between 8 and 9 million cells/kg, between 8 and 10 million cells/kg, or between 9 and 10 million cells/kg.

In one embodiment, the amount of pharmaceutical composition administered is reduced by at least 10%, at least 20%, at least 30%, at least 40%, at least 50%, at least 60%, at least 70%, at least 80%, at least 90%, at least 95% or more when administration is ICV or ITL.

In one embodiment, the dosage administered is reduced by at least 10%, at least 20%, at least 30%, at least 40%, at least 50%, at least 60%, at least 70%, at least 80%, at least 90%, at least 95% or more when administration is ICV or ITL.

The pharmaceutical composition can further comprise pharmaceutically acceptable surfactants (e.g., glycerides), excipients (e.g., lactose), carriers, serum, plasma, diluents or vehicles. In some embodiments, the pharmaceutical composition comprises a cryopreservative, e.g., DMSO.

### Administration

Methods and compositions described herein at directed at the treatment or prevention of a CNS disease or disorder, e.g., MS. In one embodiment, the composition comprising the modified hematopoietic stem cells described herein is administered to a subject that has been diagnosed with MS to treat the disease in the subject. In one embodiment, the composition described herein is administered to a subject at risk of developing MS to prevent or slow the onset of the disease in the subject. Subjects having MS can be identified by a physician using current methods of diagnosing a condition. Symptoms and/or complications of MS, which characterize these disease and aid in diagnosis are well known in the art and include but are not limited to, lesion formation; fatigue; tingling, pain or numbness of extremities; loss of balance, trouble walking; changes in vision; clinical depression; impaired cognitive function; poor muscle coordination; sexual dysfunction; slurred speech and stuttering; and bladder and bowel dysfunction. Tests that may aid in a diagnosis of, e.g. MS, include but are not limited MRI, and blood and cerebrospinal fluid testing for markers of MS. A family history of, e.g., MS, will also aid in determining if a subject is likely to have the condition or in making a diagnosis of MS.

The compositions described herein (e.g., comprising PD-L1-expressing HSCs) can be administered to a subject having or diagnosed as having a CNS disease or disorder, e.g., MS. In some embodiments, the methods described herein comprise administering an effective amount of the composition to a subject in order to alleviate at least one symptom of, e.g., MS. As used herein, “alleviating at least one symptom of MS” is ameliorating any condition or symptom associated with, e.g., MS (e.g., lesion formation; neuroinflammation; fatigue; tingling, pain or numbness of extremities; loss of balance, trouble walking; changes in vision; clinical depression; impaired cognitive function; poor muscle coordination; sexual dysfunction; slurred speech and stuttering; and bladder and bowel dysfunction). As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In one embodiment, the composition is administered systemically or locally (e.g., to a lesion e.g., on the brain or spinal cord). In one embodiment, the composition is administered intravenously. In some embodiments, the composition is administered by intrathecal or intracerebroventricular administration. In one embodiment, the composition is administered continuously, in intervals, or sporadically. The route of administration of the composition will be optimized by a skilled practitioner.

In one embodiment, the composition of PD-L1-expressing HSCs is administered locally to the CNS. In one embodiment, the composition of PD-L1-expressing HSCs is administered locally to the brain, but not the spinal cord. In one embodiment, the composition of PD-L1-expressing HSCs is administered locally to the spinal cord, but not the brain. In one embodiment, the composition of PD-L1-expressing HSCs is administered locally to the spinal cord and brain at different time points or at substantially the same time.

In one embodiment, the composition of PD-L1-expressing HSCs is administered locally to a lesion on the CNS. In one embodiment, the composition of PD-L1-expressing HSCs is administered locally to a lesion on the brain, but not the spinal cord. In one embodiment, the composition of PD-L1-expressing HSCs is administered locally to a lesion on the spinal cord, but not the brain.

In one embodiment, following administration, the modified, PDL1⁺-expressing HSCs are detected at the site of the lesion, regardless of the mode of administration. For example, the modified HSCs are detected at the site of the lesion following systemic administration.

The term “effective amount” as used herein refers to the amount of the composition that can be administered to a subject having or diagnosed as having a CNS disease or disorder, e.g., MS, needed to alleviate at least one or more symptom of, e.g., MS. The term “therapeutically effective amount” therefore refers to an amount of a composition that is sufficient to provide, e.g., a particular anti-MS effect when administered to a typical subject. An effective amount as used herein, in various contexts, would also include an amount of a composition sufficient to delay the development of a symptom of, e.g., MS, alter the course of a symptom of, e.g., MS (e.g., slowing the progression of lesion formation), or reverse a symptom of, e.g., (e.g., repair or shrink previously formed lesions). Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

### Dosage

“Unit dosage form” as the term is used herein refers to a dosage suitable for one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

Usually a daily dosage of active ingredient (e.g., of at least a second therapeutic) can be about to 500 milligrams per kilogram of body weight. Ordinarily 1 to 40 milligrams per kilogram per day given in divided doses 1 to 6 times a day or in sustained release form is effective to obtain desired results. The active ingredient will ordinarily be present in an amount of about 0.5-95% by weight based on the total weight of the composition. Second or subsequent administrations can be administered at a dosage which is the same, less than or greater than the initial or previous dose administered to the individual.

A second or subsequent administration is preferably during or immediately prior to relapse or a flare-up of the disease or symptoms of the disease, e.g., a CNS disease, such as MS. For example, second and subsequent administrations can be given between about one day to 30 weeks from the previous administration. Two, three, four or more total administrations can be delivered to the individual, as needed.

The precise dose to be employed in the formulation will also depend on the route of administration, and the seriousness and/or progression of the disease or disorder, e.g., MS, and should be decided according to the judgment of the practitioner and each patient's circumstances. Effective doses may be extrapolated from dose-response curves derived from in vitro or animal model test systems.

In one embodiment, a dose of modified hematopoietic stem cells is delivered to a subject intravenously.

In some embodiments, the dosage is at least 1×10⁴ cells per implantation. In other embodiments, the dosage is at least 5×10⁴ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 5×10⁸ cells, at least 1×10⁹ cells, at least 5×10⁹ cells, or at least 1×10¹⁰ cells or more per implantation into a subject. Second or subsequent administrations can be administered at a dosage which is the same, less than or greater than the initial or previous dose administered to the individual.

In some embodiments, the dosage of genetically modified PD-L1⁺ HSC cells administered to a recipient subject is about at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.5×10⁶ cells/kg of bodyweight, at least 0.75×10⁶ cells/kg of bodyweight, at least 1×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, or at least 30×10⁶ cells/kg of bodyweight of the subject recipient.

In some embodiments, the dosage is at least 2×10⁶ cells/kg bodyweight of the recipient subject. In other embodiments, the dosage is at least 3×10⁶ cells/kg of bodyweight, at least 4×10⁶ cells/kg of bodyweight, at least 5×10⁶ cells/kg of bodyweight, at least 6×10⁶ cells/kg of bodyweight, at least 7×10⁶ cells/kg of bodyweight, at least 8×10⁶ cells/kg of bodyweight, at least 9×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, or at least 30×10⁶ cells/kg of bodyweight of the subject recipient.

In particular embodiments, subjects receive a dose of genetically modified PD-L1⁺ HSC cells of about 1×10⁵ cells/kg of body weight of the subject recipient, about 5×10⁵ cells/kg of body weight, about 1×10⁶ cells/kg of body weight, about 2×10⁶ cells/kg of body weight, about 3×10⁶ cells/kg of body weight, about 4×10⁶ cells/kg of body weight, about 5×10⁶ cells/kg of body weight, about 6×10⁶ cells/kg of body weight, about 7×10⁶ cells/kg of body weight, about 8×10⁶ cells/kg of body weight, about 9×10⁶ cells/kg of body weight, about 1×10⁷ cells/kg of body weight, about 5×10⁷ cells/kg of body weight, about 1×10⁸ cells/kg of body weight, or more in one single intravenous dose. In certain embodiments, patients receive a dose of genetically modified PD-L1⁺ HSC cells of at least 1×10⁵ cells/kg of body weight, at least 5×10⁵ cells/kg of body weight, at least 1×10⁶ cells/kg of body weight, at least 2×10⁶ cells/kg of body weight, at least 3×10⁶ cells/kg of body weight, at least 4×10⁶ cells/kg of body weight, at least 5×10⁶ cells/kg of body weight, at least 6×10⁶ cells/kg of body weight, at least 7×10⁶ cells/kg of body weight, at least 8×10⁶ cells/kg of body weight, at least 9×10⁶ cells/kg of body weight, at least 1×10⁷ cells/kg of body weight, at least 5×10⁷ cells/kg of body weight, at least 1×10⁸ cells/kg of body weight, or more in one single intravenous dose.

In an additional embodiment, subjects receive a dose of modified hematopoietic stem cells of about 1×10⁵ cells/kg of body weight to about 1×10⁸ cells/kg of body weight, about 1×10⁶ cells/kg to about 1×10⁸ cells/kg, about 1×10⁶ cells/kg to about 9×10⁶ cells/kg, about 2×10⁶ cells/kg to about 8×10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

In various embodiments, the methods of the invention provide more robust and safe gene therapy than existing methods and comprise administering a population or dose of hematopoietic stem cells comprising about 5% genetically modified PD-L1⁺ HSC cells, about 10% genetically modified PD-L1⁺ HSC cells, about 15% genetically modified PD-L1⁺ HSC cells, about 20% genetically modified PD-L1⁺ HSC cells, about 25% genetically modified PD-L1⁺ HSC cells, about 30% genetically modified PD-L1⁺ HSC cells, about 35% genetically modified PD-L1⁺ HSC cells, about 40% genetically modified PD-L1⁺ HSC cells, about 45% genetically modified PD-L1⁺ HSC cells, or about 50% genetically modified PD-L1⁺ HSC cells, to a subject. In some embodiments, the method comprises administering a population or dose of hematopoietic stem cells comprising at least 50% genetically modified PD-L1⁺ HSC cells, at least 60% genetically modified PD-L1⁺ HSC cells, at least 70% genetically modified PD-L1⁺ HSC cells, at least 80% genetically modified PD-L1⁺ HSC cells, at least 90% genetically modified PD-L1⁺ HSC cells, at least 95% genetically modified PD-L1⁺ HSC cells, or at least 98% genetically modified PD-L1⁺ HSC cells.

### Combination Therapy

In one embodiment, the composition described herein is used as a monotherapy. In one embodiment, the composition described herein is not used in combination with any other known agents and therapies for a CNS disease or disorder, e.g., MS. That is, administration of the composition specifically excludes administration of at least a second agent or therapy.

In one embodiment, the composition described herein can be used in combination with other known agents and therapies for a CNS disease or disorder, e.g., MS.

Administered “in combination,” as used herein, means that two (or more) different treatments are delivered to the subject during the course of the subject's affliction with the disorder, e.g., the two or more treatments are delivered after the subject has been diagnosed with the disorder or disease (e.g., MS) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The composition described herein and the at least one additional therapy can be administered simultaneously, in the same or in separate compositions, or sequentially. For sequential administration, the composition described herein can be administered first, and the additional agent can be administered second, or the order of administration can be reversed. The composition and/or other therapeutic agents, procedures or modalities can be administered during periods of active disorder, or during a period of remission or less active disease. The composition can be administered before another treatment, concurrently with the treatment, post-treatment, or during remission of the disorder.

Exemplary therapeutics used to treat MS, and that may be used in combination with the composition of PD-L1+-HSCs described herein include, but are not limited to, interferon class, IFN-β-1a (REBIF® and AVONEX®) and IFN-β-1b (BETASERON®); glatiramer acetate (COPAXONE®) polypeptide; natalizumab (TYSABRI®); dimethyl fumarate (TECFIDERA®), fingolimod (GILYENYA®), and mitoxantrone (NOVANTRON®), cytotoxic agent. Other drugs, including corticosteroids, methotrexate, cyclophosphamide, azathioprine, and intravenous (IV) immunoglobulins have been used with varying degrees of success. The above listed drugs are approved for treatment of RRMS. Ocrelizumab (OCREVUS®) has been approved for the treatment of primary-progressive MS.

Various other treatments for MS that can be used in combination with the compositions of PD-L1+-HSCS described herein are known in the art, and include i.v. steroids (e.g., bethamethasone (Celestone), prednisone (Prednisone Intensol), prednisolone (Orapred, Prelone), triamcinolone (Aristospan Intra-Articular, Aristospan Intralesional, Kenalog), and methylprednisolone (Medrol, Depo-Medrol, Solu-Medrol)); glucocorticoids; off-label drugs (e.g., Amantadine (Osmolex ER®), Azathioprine (Imuran®), Cyclophosphamide, Duloxetine (Cymbalta®), Methotrexate, Minocycline, Mycophenolate mofetil (CellCept®). Pregabalin (Lyrica®), Rituximab (Rituxan®); and Simvastatin (Zocor®)); deep brain stimulation; plasmapheresis; physical therapy; occupational therapy; Botulinum toxin; stem cell therapy; Wahls protocol diet; and alternative therapies (e.g., acupuncture, yoga, relaxation, herbal remedies, and massage).

When administered in combination, the composition and the additional agent (e.g., second or third agent), or all, can be administered in an amount or dose that is higher, lower or the same as the amount or dosage of each used individually, e.g., as a monotherapy. For example, the composition and the additional agent can be administered at sub-therapeutic doses when administered in combination. In certain embodiments, the administered amount or dosage of the composition, the additional agent (e.g., second or third agent), or all, is lower (e.g., at least 20%, at least 30%, at least 40%, or at least 50%) than the amount or dosage of each used individually. In other embodiments, the amount or dosage of composition, the additional agent (e.g., second or third agent), or all, that results in a desired effect (e.g., treatment of a CNS disease or disorder, such as MS) is lower (e.g., at least 20%, at least 30%, at least 40%, or at least 50% lower) than the amount or dosage of each individually required to achieve the same therapeutic effect.

### Parenteral Dosage Forms

Parenteral dosage forms of a composition described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Routes of administration include, but are not limited to intravenous, intrathecal, intracerebroventricular, or any therapeutically efficacious route of administration.

In one embodiment, administration is intravenous administration, intrathecal administration, or intracerebroventricular administration.

In one embodiment, administration is intravenous administration.

In one embodiment, administration is intrathecal administration. As used herein, the term “intrathecal administration” is intended to include delivering the therapeutic formulation containing the therapeutic composition of the invention (e.g. a composition comprising PD-L1⁺-expressing HSCs) directly into the cerebrospinal fluid of a subject, by techniques including lateral cerebroventricular injection through a burrhole or cisternal or lumbar puncture or the like (described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head.

In one embodiment, administration is intracerebroventricular administration, e.g., administered directly into the CNS.

Therapeutic compositions or pharmaceutical compositions can be formulated for passage through the blood-brain barrier or direct contact with the endothelium. The modified hematopoietic stem cells, and the compositions described herein can be administered by any known route. By way of example, the modified hematopoietic stem cells and the compositions described herein can be administered by a mucosal, pulmonary, topical, or other localized or systemic route (e.g., enteral and parenteral). The modified hematopoietic stem cells may be administered by any convenient route, for example by infusion or bolus injection, and may be administered together with other biologically active agents.

### Efficacy

The efficacy of a composition described herein, e.g., for the treatment of MS, can be determined by the skilled practitioner. However, a treatment is considered “effective treatment,” as the term is used herein, if one or more of the signs or symptoms of, e.g., MS, are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., lesion formation; fatigue; tingling, pain or numbness of extremities; loss of balance, trouble walking; changes in vision; clinical depression; impaired cognitive function; poor muscle coordination; sexual dysfunction; slurred speech and stuttering; and bladder and bowel dysfunction. Efficacy can also be measured by a failure of an individual to worsen as assessed by hospitalization, or need for medical interventions (i.e., progression of numbness of extremities, loss of balance, formation of lesions, etc.). Methods of measuring these indicators are known to those of skill in the art and/or are described herein.

Efficacy can be assessed in animal models of a condition described herein, for example, a mouse model or an appropriate animal model of a CNS disease or disorder, e.g., MS, as the case may be. When using an experimental animal model, efficacy of treatment is evidenced when a statistically significant change in a marker is observed, e.g., decreased numbness of extremities, increased balance and ease of walking, decreased quantity of new lesions, etc.

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean ±1%.

All patents and publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosures or for any other reason. All statements as to the date or representation as to the contents of these documents is based on the information available to the applicants and does not constitute any admission as to the correctness of the dates or contents of these documents.

The invention described herein can be further defined by any of the following numbered paragraphs:
1. A method of treating a central nervous system (CNS) disease or disorder, the method comprising administering to a subject diagnosed with the CNS disease or disorder a composition comprising a population of genetically modified, programmed cell death-1 receptor ligand (PD-L1)⁺-expressing hematopoietic stem cells (HSCs),
   wherein the CNS disease or disorder involves inflammation of the CNS.
2. The method of paragraph 1, wherein the CNS disease or disorder is selected from the group consisting of multiple sclerosis (MS), Systemic lupus erythematosus (SLE), inflammatory brain disease, inflammation of the CNS, central nervous system vasculitis, Neuromyelitis Optica Spectrum Disorder.
3. The method of any of the preceding paragraphs, wherein the CNS disease or disorder is MS.
4. The method of any of the preceding paragraphs, wherein the MS is relapsing remitting MS (RRMS).
5. The method of any of the preceding paragraphs, wherein the MS is secondary progressing MS (SPMS).
6. The method of any of the preceding paragraphs, wherein the MS is primary progressive MS (PPMS).
7. The method of any of the preceding paragraphs, wherein the MS is non-active, active, highly active (HA), or rapidly evolving severe relapsing remitting MS (RE).
8. The method of any of the preceding paragraphs, wherein the CNS disease or disorder is inflammation of the CNS.
9. The method of any of the preceding paragraphs, wherein inflammation of the CNS is inflammation of the spinal cord.
10. The method of any of the preceding paragraphs, wherein inflammation of the CNS is inflammation of the brain.
11. The method of any of the preceding paragraphs, wherein inflammation of the CNS is inflammation of the spinal cord and brain.
12. The method of any of the preceding paragraphs, further comprising the step, prior to administering, of diagnosing the subject as having CNS disease or disorder.
13. The method of any of the preceding paragraphs, further comprising the step, prior to administering, of receiving the results of an assay that diagnoses a subject as having CNS disease or disorder.
14. The method of any of the preceding paragraphs, further comprising the step, prior to administering, of diagnosing the subject as having MS.
15. The method of any of the preceding paragraphs, further comprising the step, prior to administering, of receiving the results of an assay that diagnoses a subject as having MS.
16. The method of any of the preceding paragraphs, further comprising the step, prior to administering, of diagnosing the subject as having inflammation of the CNS.
17. The method of any of the preceding paragraphs, further comprising the step, prior to administering, of receiving the results of an assay that diagnoses a subject as having inflammation of the CNS.
18. The method of any of the preceding paragraphs, wherein the PD-L1⁺-expressing HSCs carry an exogenous copy of a nucleic acid encoding a programmed cell death-1 receptor ligand (PD-L1).
19. The method of any of the preceding paragraphs, wherein the nucleic acid is a complementary DNA (cDNA).
20. The method of any of the preceding paragraphs, wherein the cDNA has the sequence of SEQ ID NO: 1.
21. The method of any of the preceding paragraphs, wherein the cDNA has the sequence of SEQ ID NO: 2.
22. The method of any of the preceding paragraphs, wherein the nucleic acid is a genomic DNA.
23. The method of any of the preceding paragraphs, wherein the nucleic acid is integrated into the genome of the cells.
24. The method of any of the preceding paragraphs, wherein the nucleic acid has been introduced into the cells via a vector.
25. The method of any of the preceding paragraphs, wherein the vector is a viral vector or non-viral vector.
26. The method of any of the preceding paragraphs, wherein the viral vector is a lentiviral vector.
27. The method of any of the preceding paragraphs, wherein the PD-L1⁺-expressing HSCs are mammalian HSCs or human HSCs.
28. The method of any of the preceding paragraphs, wherein the PD-L1⁺-expressing HSCs are obtained by genetically modifying HSCs obtained from bone marrow, umbilical cord, amniotic fluid, chorionic villi, cord blood, placental blood or peripheral blood.
29. The method of any of the preceding paragraphs, wherein the PD-L1⁺-expressing HSCs are obtained by genetically modifying HSCs obtained from mobilized peripheral blood.
30. The method of any of the preceding paragraphs, further comprising the step, prior to administering, of obtaining HSCs from bone marrow, umbilical cord, amniotic fluid, chorionic villi, cord blood, placental blood or peripheral blood, and genetically modifying the obtained HSCs by introducing an exogenous copy of a nucleic acid encoding a PD-L1.
31. The method of any of the preceding paragraphs, wherein the obtained HSC cells are ex vivo cultured before, or after, or both before and after the introduction of the exogenous copy of a nucleic acid encoding a PD-L1.
32. The method of any of the preceding paragraphs, wherein the HSCs are derived from a healthy individual.
33. The method of any of the preceding paragraphs, wherein the HSCs are derived from an individual with a diagnosed disease or disorder.
34. The method of any of the preceding paragraphs, wherein the diagnosed disease or disorder is a CNS disease or disorder.
35. The method of any of the preceding paragraphs, wherein the CNS disease or disorder is MS.
36. The method of any of the preceding paragraphs, wherein the CNS disease or disorder is inflammation of the CNS.
37. The method of any of the preceding paragraphs, wherein the HSCs are derived from the subject.
38. The method of any of the preceding paragraphs, wherein the population of PD-L1⁺-expressing HSCs are autologous, allogeneic, or xenogeneic to the subject.
39. The method of any of the preceding paragraphs, wherein the PD-L1⁺-expressing HSCs are produced by a method comprising:
   a) contacting a population of unmodified HSCs with a vector carrying an exogenous copy of a nucleic acid encoding a PD-L1, thereby obtaining modified HSCs;
   b) ex vivo culturing the resultant modified cells from the contacting; and
   c) establishing expression of PD-L1 on the modified HSCs, thereby producing a population of modified HSCs cells expressing PD-L1.
40. The method of any of the preceding paragraphs, wherein the method further comprises establishing that the population of modified HSCs have an at least 1-fold, 2-fold, 3-fold, 4-fold, or more percent increase in the number of PD-L1 expressing HSCs compared to the population of unmodified HSCs.
41. The method of any of the preceding paragraphs, wherein administration is systemic.
42. The method of any of the preceding paragraphs, wherein administration is local administration to at least a lesion, the brain, or the spinal cord.
43. The method of any of the preceding paragraphs, wherein the lesion is a site of nerve cell damage.
44. The method of any of the preceding paragraphs, wherein the lesion is present on the brain or spinal cord.
45. The method of any of the preceding paragraphs, administering reduces, delays, or stops the progression of MS.
46. The method of any of the preceding paragraphs, administering reduces or eliminates inflammation of the CNS.
47. The method of any of the preceding paragraphs, administering reduces or eliminates the population of neutrophils in the CNS.
48. The method of any of the preceding paragraphs, administering increases the population of CD3+ T cells in the CNS.
49. The method of any of the preceding paragraphs, wherein the PD-L1⁺-expressing HSCs are detected at lesions in the subject following administration.
50. The method of any of the preceding paragraphs, further comprising administering at least one additional therapeutic.
51. The method of any of the preceding paragraphs, wherein the at least one additional therapeutic is an anti-MS therapeutic.
52. A method of treating a CNS disease or disorder associated with inflammation of the CNS in a subject comprising:
   a) providing a population of unmodified hematopoietic stem cells (HSCs);
   b) contacting the population of unmodified HSCs of (a) with a vector carrying an exogenous copy of a nucleic acid encoding a PD-L1, thereby obtaining modified HSCs;
   c) ex vivo culturing the resultant modified HSCs from the contacting;
   d) establishing expression of PD-L1 on the modified HSCs, thereby producing a population of PD-L1⁺-expressing HSCs; and
   e) transplanting said population of PD-L1⁺-expressing HSCs into a recipient subject having a CNS disease or disorder associated with inflammation of the CNS, thereby treating the CNS disease or disorder in the recipient subject.
53. A method of treating MS in a subject comprising:
   a) providing a population of unmodified hematopoietic stem cells (HSCs);
   b) contacting the population of unmodified HSCs of (a) with a vector carrying an exogenous copy of a nucleic acid encoding a PD-L1, thereby obtaining modified HSCs;
   c) ex vivo culturing the resultant modified HSCs from the contacting;
   d) establishing the expression of PD-L1 on the modified HSCs, thereby producing a population of PD-L1⁺-expressing HSCs; and
   e) transplanting said population of PD-L1⁺-expressing HSCs into a recipient subject having MS, thereby treating MS in the recipient subject.
54. A method of treating or preventing inflammation of the CNS in a subject comprising:
   a) providing a population of unmodified hematopoietic stem cells (HSCs);
   b) contacting the population of unmodified HSCs of (a) with a vector carrying an exogenous copy of a nucleic acid encoding a PD-L1, thereby obtaining modified HSCs;
   c) ex vivo culturing the resultant modified HSCs from the contacting;
   d) establishing expression of PD-L1 on the modified HSCs, thereby producing a population of PD-L1⁺-expressing HSCs; and
   e) transplanting said population of PD-L1⁺-expressing HSCs into a recipient subject having or at risk of having inflammation of the CNS, thereby preventing or treating inflammation of the CNS in the recipient subject.
55. A method of treating a CNS disease or disorder associated with inflammation of the CNS in a subject comprising:
   a) receiving a population of PD-L1⁺-expressing HSCs; and
   b) transplanting said population of PD-L1⁺-expressing HSCs into a recipient subject having a CNS disease or disorder associated with inflammation of the CNS, thereby treating the CNS disease or disorder in the recipient subject.
56. A method of treating MS in a subject comprising:
   a) receiving a population of PD-L1⁺-expressing HSCs; and
   b) transplanting said population of PD-L1⁺-expressing HSCs into a recipient subject having MS, thereby treating MS in the recipient subject.
57. A method of treating or preventing inflammation of the CNS in a subject comprising:
   a) receiving a population of PD-L1⁺-expressing HSCs; and
   b) transplanting said population of PD-L1⁺-expressing HSCs into a recipient subject having or at risk of having inflammation of the CNS, thereby preventing or treating inflammation of the CNS in the recipient subject.
58. The method of any of the preceding paragraphs, wherein the population of HSCs autologous to the recipient subject.
59. The method of any of the preceding paragraphs, wherein the population of HSCs allogeneic to the recipient subject.
60. The method of any of the preceding paragraphs, wherein the population of HSCs is xenogeneic to the recipient subject.
61. A population of PD-L1⁺-expressing HSCs, comprising HSCs obtained from a subject diagnosed with a CNS disease or disorder and an exogenous copy of a nucleic acid encoding PD-L1.
62. The population of PD-L1⁺-expressing HSCs of any of the preceding paragraphs, wherein the exogenous copy of the nucleic acid encoding PD-L1 is integrated into a genome of the HSCs.
63. The population of PD-L1⁺-expressing HSCs of any of the preceding paragraphs, wherein the CNS disease or disorder is MS.
64. A pharmaceutical composition comprising the population of PD-L1⁺-expressing HSCs of any of the preceding paragraphs in a physiologically acceptable excipient.
65. An ex vivo method of producing a population of PD-L1⁺-expressing hematopoietic stem cells (HSCs), the method comprising:
   a) obtaining a population of unmodified HSCs from a subject diagnosed with a CNS disease or disorder;
   b) contacting the population of unmodified HSCs with a vector carrying an exogenous copy of a nucleic acid encoding PD-L1, thereby obtaining modified HSCs;
   c) ex vivo culturing the resultant modified HSCs; and
   d) establishing expression of PD-L1 on the modified HSCs, thereby producing a population of modified HSCs expressing PD-L1.
66. The method of any of the preceding paragraphs, wherein in step a), the population of unmodified HSCs are obtained from the bone marrow, umbilical cord, amniotic fluid, chorionic villi, cord blood, placental blood or peripheral blood of the subject.
67. The method of any of the preceding paragraphs, wherein the CNS disease or disorder is MS.
68. The method of any of the preceding paragraphs, wherein administering is intravenous administration, intrathecal administration, or intracerebroventricular administration.

### EXAMPLES

### Example 1—Material and Methods Relating to Examples 2 and 3

Multiple sclerosis (MS) studies were carried out in the myelin oligodendrocyte glycoprotein (MOG) experimental autoimmune encephalitis (EAE) animal model (MOG-EAE). Although there are differences between the pathophysiology of EAE and the human disease, EAE has become a very powerful and often utilized animal model for the development of new MS therapies (Constantinescu et al. 2011). In particular, because of the inflammatory nature of EAE and the autoimmune contribution to the disease, EAE has been mostly useful in determining the efficacy of immunomodulatory treatments (Robinson et al. 2014). Typical features of EAE disease include the primary demyelination of axonal tracks, with impaired axonal conduction in the central nervous system (CNS), and progressive hind-limb paralysis. However, there are many pathophysiologic forms of EAE with varying clinical presentation patterns, depending on the animal species and strain, priming protein/peptide, and route of immunization employed.

The EAE model has been proven to be a reliable model of human MS, demonstrating its utility and reliability over many years. There are eminent reviews and papers about the use of the EAE model (e.g., Baxter, Nat Rev Immunol, 2007; Constantinescu, B J P, 2011; Pluchino, Nature, 2003, and Pluchino, Nature, 2005), which enables the in vivo evaluation of strengths and weaknesses of an experimental approach. As a matter of fact, the EAE model has been and continues to be the model of choice for drug and ATMP development.

Data presented herein investigate transplantation of murine HSPCs transduced with a lentiviral vector (LV) to express PD-L1 as a therapeutic approach to modify disease manifestation in the EAE model. The method of genetically modifying HSCs by introducing LV to express PD-L1 is described in international patent application no. WO2017/015320, which is incorporated by reference in its entirety herein.

### Mice Studies

All experiments were performed on C57BL/6 CD45.2 (recipient) and C57BL/6 CD45.1 (donor) mice in accordance with the local institutional animal care and use committee of Boston Children's Hospital and Dana Farber Cancer Institute. The CD45.1/CD45.2 chimerism was used to track the distribution of donor-derived cells in the different tissues.

### EAE Induction and HSPC Transplantation

EAE was induced in 12 C57BL/6 CD45.2 12-week-old female mice using an emulsion of Freund's complete adjuvant (CFA) and MOG35-55 (Hooke Laboratories, Lawrence, MA), as described (Paterson et al. J Exp Med 2015). Briefly, at day 0, 100 μl of a 1:1 emulsion of CFA and 1 mg/mL MOG33-55 were injected subcutaneously (sc) into each flank. Within 2 hours, 400 ng of Pertussis toxin were administered intraperitoneally (ip); 24 hours later, the Pertussis toxin administration was repeated.

At day 1, a first group of mice (n=7) received a single intravenous (iv) injection of 3×10⁶ of LV-transduced PD-L1-expressing HSPCs (LvPDL1) isolated from the bone marrow (BM) of C57BL/6/CD45.1 mice, while a second group of mice was left untreated (n=5). All mice were monitored daily, weighed and scored for clinical symptom development, as outlined in Table 1 (Miller at el. Current Protocols in Immunology 2007). Upon occurrence of severe symptoms animals were provided with food within the cage and gel packs to ensure access to fluids. Mice were sacrificed at day 30 post immunization for immune phenotyping. Brain, spinal cord, spleen and bone marrow were collected.

TABLE 1
grading system for clinical assessment of EAE

| SCORE | CLINICAL SIGNS |
|---|---|
| 0 | Normal mouse; no overt signs of disease |
| 1 | Limp tail (a) or hind limb weakness but not both |
| 2 | Limp tail (a) and hind limb weakness (b) (paraparesis) |
| 3 | Partial hind limb paralysis (c) (paraplegia) |
| 4 | Complete hind limb paralysis (d) (quadriparesis) |
| 5 | Moribund state; paralysis of all limbs (quadriplegia); |
| | death by EAE: sacrifice for human reasons |

Table 1 shows a grading system for clinical assessment of RAE (Miller et al. Current Protocols in immunology 2007). ^{(a)} Limp tail: complete flaccidity of the tail, and absence of curling at the tip of the tail when mouse is picked up. ^{(b)} Hind limb weakness: observed as a waddling gait, the objective sign being that, in walking, mouse's hind limbs fall through the wire cage tops. ^{(c)} Partial hind limb paralysis: mouse can no longer use hind limbs to maintain rump posture or walk but can still move one or both limbs to some extent. ^{(d)} Complete hind limb paralysis: total loss of movement in hind limbs; mouse drags itself only on its forelimbs. Mice at this stage are given food on the cage floor, long sipper tubes, and daily subcutaneous saline injections to prevent death by dehydration.

### Vector Production

Full-length cDNA encoding murine PD-L1 was cloned into the transfer plasmid pHAGE-fullEF1a-TRE-IZsGreen, a non-replicative lentivirus (LV) containing internal EF1a promoter, IRES-ZsGreen (IRES-fluorescent tag), and Tet-responsive element. Expression of mPDL1 and ZsGreen was induced in the presence of doxycycline. The expression of the bicistronic transcript was driven by the human elongation factor 1 alpha (EF1α) promoter. VSV-G pseudotyped LVs were generated by co-transfection of the murine PD-L1 transfer plasmid together with the packaging expression plasmids (Gag/Pol, Tat, Rev) along with the envelope expressing plasmid encoding for VSV-G into 293T cells using the TransIT-293 transfection reagent (Minis Bio, Madison, WI). Starting at 24 hour post transfection, the supernatant containing the viral particles was collected, centrifuged at 1800 rpm for 5 minutes to remove dead cells and debris, and concentrated using the Lenti-X concentrator, following the manufacturer's protocol (Clontech, Mountain View, CA). Viral stocks were stored at ≤−65° C. until transduction experiments were performed.

Two batches of murine PD-L1 LV were produced and titered: batch #1: 8.41×10⁷ transducing units (TU)/ml; and batch #2: 1.74×107 TU/ml.

KL isolation and LV transduction—Murine HSPCs were harvested from C57BL6 CD45.1 mice aged between 6 and 8 weeks. Mice were euthanized with CO₂ and BM was harvested upon crushing from femur, tibia and humerus with phosphate-buffered saline (PBS) plus 2% fetal bovine serum (FBS). Red blood cells were lysed using Ammonium-Chloride-Potassium buffer (Quality Biological, Gaithersburg, MD) at the concentration of 10⁸ cells/ml. Bone marrow cells were then depleted of lineage positive (Lin+) cells using the lineage cell depletion kit according to the manufacturer's instructions (Miltenyi Biotec, Bergisch Gladbach, Germany).

To isolate c-kit+ cells, the lineage negative (Lin−) cells were selected for c-kit expression using CD117 microbeads (Miltenyi Biotec, Bergisch Gladbach, Germany) and isolating the positive fraction according to the manufacturer's instructions. Isolated murine c-Kit+Lin− (KL) cells were thereafter transduced with LV encoding for murine PD-L1 at MOI 0.17 in StemSpan™ serum-free expansion medium II (Stemcell Technologies, Cambridge, MA) in the presence of 2 ng/mL polybrene (Sigma-Aldrich, St. Louis, MO), 10 ng/ml stem cell factor (Miltenyi Biotec, Bergisch Gladbach, Germany) and 100 ng/ml thrombopoietin (Miltenyi Biotec, Bergisch Gladbach, Germany). Twenty-four hours after transduction, cells were washed twice with PBS 1× and collected for flow cytometry and molecular analyses to further control the PD-L1 expression. Before transplant, cells were resuspended at a concentration of 3×10⁶/100 μl PBS and were administered via the tail vein. Adoptively transferred mice were then administered with doxycycline in water (50 mg/ml), changed every 72 hours, to induce expression of PD-L1 in the transduced HSPCs.

### Tissue Collection and Processing for Flow Cytometry and Histology

According to the time and the group of animals described, mice under deep anesthesia were euthanized at day 30 by extensive intracardiac perfusion with cold PBS over 15 min after clumping the femur. Organs were then collected and differentially processed, as described herein below.

BM cells were harvested by flushing the tibias with PBS plus 2% FBS. After spinning at 1500 rpm for 5 min at room temperature cells were re-suspended at a concentration of 1×10⁷ cells/ml in blocking solution (PBS, 5% FBS, 2% bovine serum albumin) and incubated for 20 min at 4° C. to block non-specific antibody binding. Cells were then incubated for 20 min at 4° C. with specific antibodies (CD45.1 PE, CD45.2 PB) to assess donor chimerism. Cell pellets were frozen and kept for vector copy number (VCN) analysis and additional biochemical assay.

Brain was removed, and the two hemispheres were differently processed. For immunofluorescence analysis, one hemisphere was cut into 2 mm-thick slices and fixed for 24 hours in 4% paraformaldehyde (PFA), embedded in optimal cutting temperature compound, and stored at less than 65° C., after equilibration in sucrose gradients (from 10 to 30%). For flow cytometry analysis, brain cells were obtained by mechanic disaggregation of one brain hemisphere in 1.5 ml in Earle's balanced salt solution (EBSS) medium and processed with papain-based digestion procedure according to the neural tissue dissociation kit (Miltenyi Biotec, Bergisch Gladbach, Germany). After washing with EBSS medium, the digested suspension was enriched in myeloid cells with Percoll gradient. Cell suspension was then washed with PBS plus 2% FBS, put in blocking solution and stained with specific antibodies in order to evaluate engraftment (CD45.1 PE, CD45.2 PB, CD11b APC) and the infiltration of inflammatory cells (CD45 PE, Ly6G APC/Cy7, CD11b A700, CD11c, NK1.1 PE Cy7; CD3 APC, B220 BV605).

Spinal cord was removed and divided in two halves longitudinally. As for the brain, 2 mm-thick slices were fixed in PFA 4%, and samples were either frozen for molecular analysis (VCN) or immediately processed for flow cytometry, as described for the brain tissue.

### Example 2—PD-L1 Expressing HSPCs Reduce the Severity of Disease Manifestations and Significantly Decrease Neuroinflammation

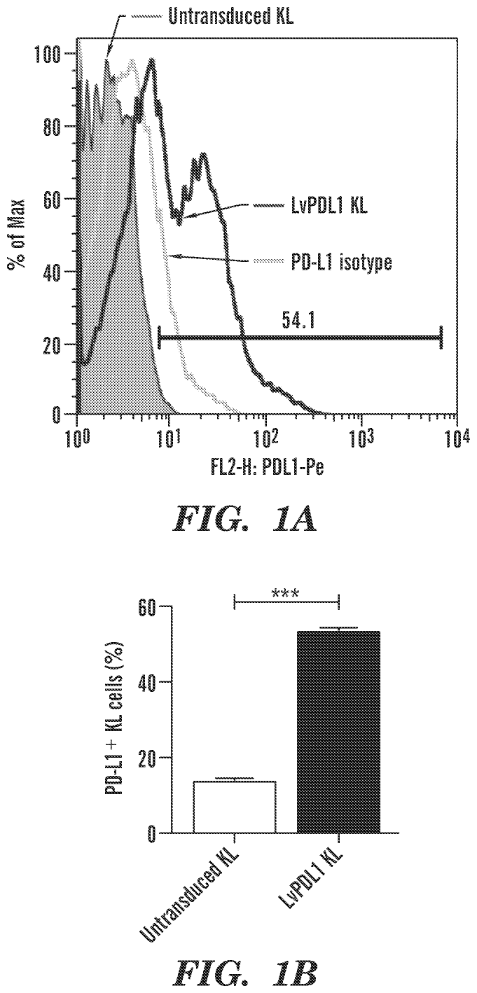

In vitro assessment—At 24 hours following LV transduction, flow cytometry analysis confirmed that the expression of PD-L1 was significantly increased in LV-transduced PD-L1-expressing KL cells (LvPDL1 KL), as shown in FIGS. 1A and 1B.

In vivo assessment—As described herein above, MOG35-55 induces EAE, which is a widely used animal model that phenocopies human MS, in the C57BL/6 mouse model. C57BL/6 mice received MOG35-55 immunization challenge at day 0. The clinical score was measured according to the grading system for clinical assessment of EAE (see Table 1; Miller et al. Current Protocols in Immunology 2007). The treatment group received 3×10⁶ LV-transduced PD-L1-expressing KL cells at day 1. All C57BL/6 mice were followed-up daily until day 30, defined herein as time of sacrifice.

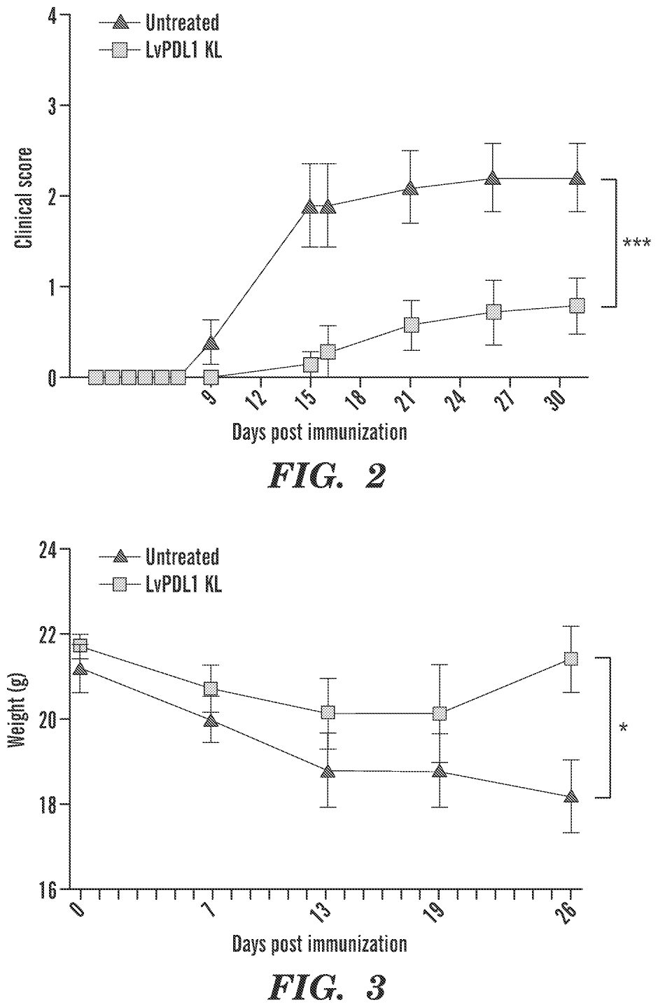

Control mice, which were challenged with MOG35-55 and received no treatment at day 1, develop progressive motor deficits that manifested at day 14 and increased in severity at day 30. In contrast, animals that received treatment with LvPDL1 KL following immunization, showed no or slowed clinical progression, and developed only mild signs of clinical EAE (FIG. 2).

Consistently, control mice showed a constant decrease in body weight over time, whereas the body came back to baseline levels in mice treated with PD-L1-expressing KL cells (P<0.05 at day 26), as shown in FIG. 3.

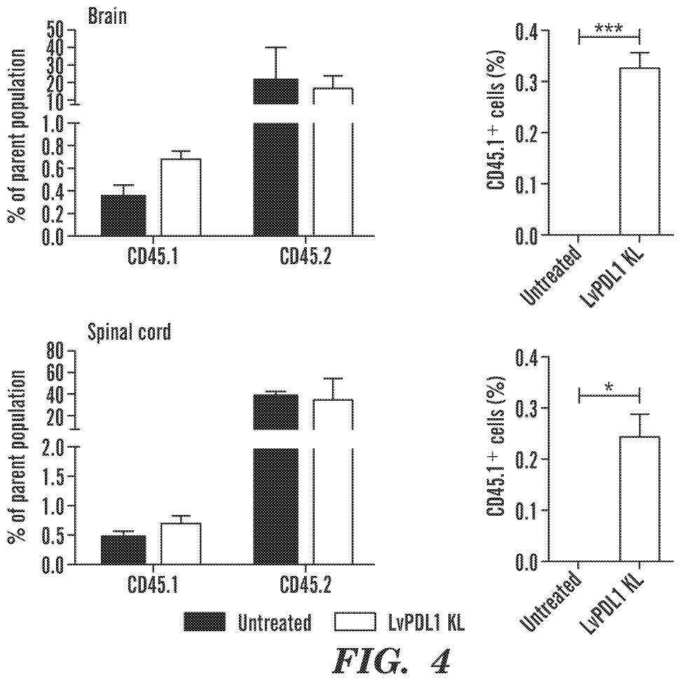

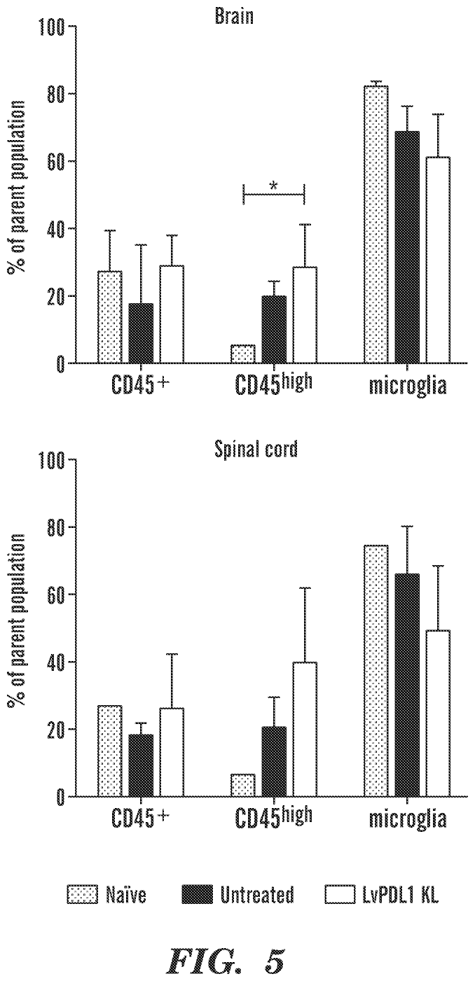

Interestingly, CD45.1+ donor derived cells were detected at lesion sites (FIG. 4). The detection of CD45.1+ donor derived cells at inflammation sites, including brain and spinal cord, indicated that PD-L1-expressing cells home and persist at EAE lesion sites following a single injection of LvPDL1 KL (FIG. 4). Conversely, no CD45.1+ cells were retrieved in either the peripheral blood or in the bone marrow of transplanted EAE mice at day 30 (data not shown).

### Example 3—PD-L1 Expressing HSPCs Contribute to Reprogramming the Immune Response

To assess whether the tested treatment affects the distribution of CNS infiltrating leukocytes, analysis of myeloid and leukocyte subpopulations was performed in the brain and spinal cord of naïve mice and of MOG35-55-immunized animals, either receiving or not receiving LvPDL1 KL at day 1 (FIGS. 5-7). The analysis on myeloid CNS subpopulations, which is identified according to the level of expression of CD45 and CD11b, showed no significant differences in CD45+ cells and microglia among groups of animals, whereas an increase of the proportion of macrophages (CD45high, CD11b+) in EAE MOG35-55 immunized mice was observed (FIG. 5). These data indicate that an expansion of inflammatory cells in the affected regions occurs in response to disease induction.

Macrophages play a dual role in MS pathology—on one hand, exerting a neuroprotective role and growth promoting effects, and on the other hand, contributing to tissue damage by production of inflammatory mediators. The dual role of macrophages can be explained by the fact that macrophages are not a single homogeneous population. Instead, several different phenotypical and functional subpopulations exist as a result of their activation status, which is influenced by environmental signals. The two most polarized phenotypes are classically activated (M1) with cytotoxic and pro-inflammatory properties and the alternatively activated (M2) macrophages, which are involved in tissue repair by producing extracellular matrix molecules and anti-inflammatory cytokines (Vogel et al. J Neuroinflammation 2013).

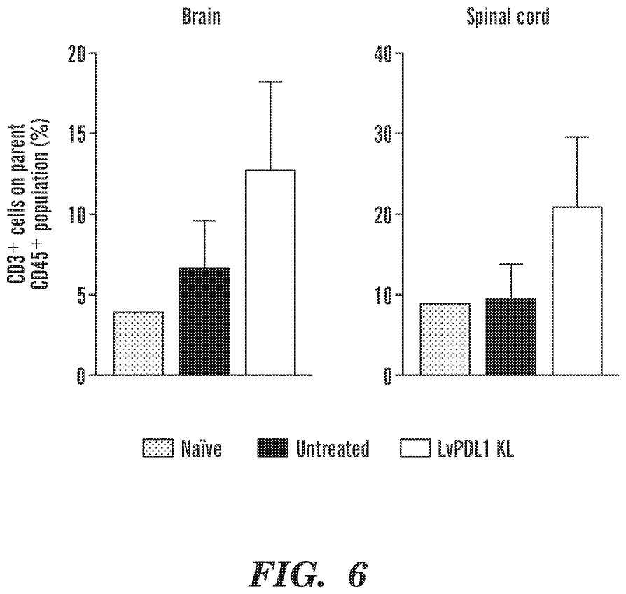

Overall, it is worth noting that a myeloid-mediated immune response was present in the LvPDL1 KL mice (FIG. 5). Consistently, an increase in the proportion of T cells, which is identified according to the expression of CD3 within the total CD45+ hematopoietic compartment, was observed in the LvPDL1 KL group in both brain and spinal cord (FIG. 6).

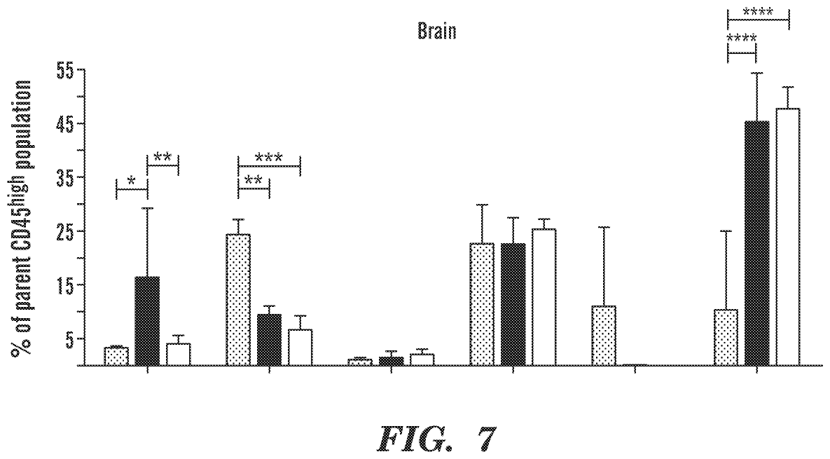

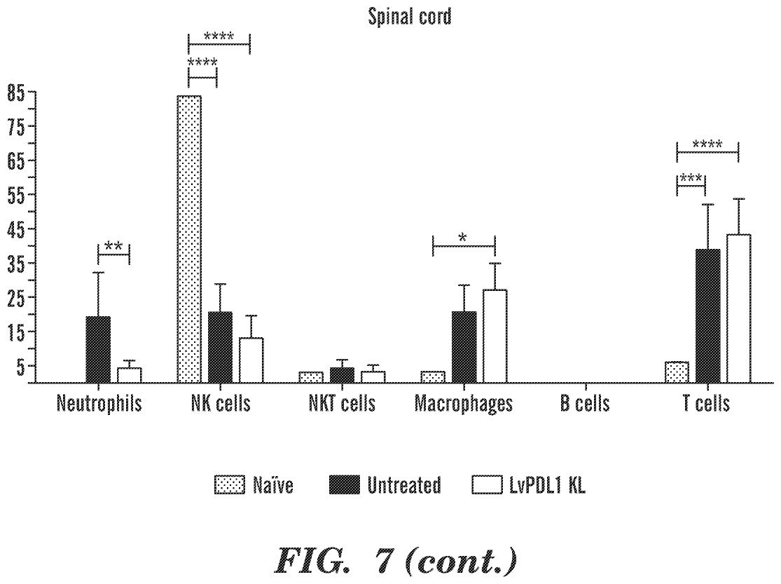

Furthermore, flow cytometry analysis found that the expansion of T cells is of a much greater magnitude than CD45high cells (FIG. 7). The increase in the proportion of total T cells, in both brain and spinal cord, was statistically significant in both EAE mouse groups as compared to naïve animals, confirming the presence of an immune response following the immunization challenge.

The proportion of NK cells in both EAE mouse groups was significantly reduced as compared to that assessed in naïve animals (FIG. 7). The control of autoreactive cells, thus preventing the onset of autoimmune manifestations, is the task of specialized immune cell subsets, called regulatory cells. The best characterized regulatory immune cell populations belong to the adaptive immune system and include regulatory T cells, type-1 regulatory T cells, and regulatory B cells. However, there is increasing evidence that the innate immune system also plays an important role in controlling autoreactive cells. There is conflicting data on a beneficial versus detrimental role of NK cells in EAE and studies on regulatory NK cells in mice are difficult to translate into humans (Gross et al. Front Immunol 2016).

A statistically significant increase in the proportion of neutrophils was observed in EAE mice that did not receive transplant with LvPDL1 KL as compared to the other animal groups (FIG. 7). The increase in neutrophil counts in EAE was consistent with the fact that neutrophils provide local cofactors in the CNS that are required for the maturation of myeloid cells into professional APCs representing an essential step for the local restimulation of myelin-specific T cells, thus promoting neuroinflammation (Steinbach et al. J Immunol 2013). Most importantly, in EAE mice receiving LvPDL1 KL the proportion of neutrophils in the brain was comparable to that measured in naïve animals, indicating the ability of LvPDL1 KL to significantly prevent the extent of neuroinflammation observed in EAE mice left untreated. This indicated that a single administration of modified, PD-L1⁺-expressing HPSCs contributed in reprogramming the autoreactive immune response into a de novo self-tolerant immune repertoire via the modulation of PD-L1 expression. This was consistent with the clinical response observed in animals undergoing treatment with LvPDL1 KL.

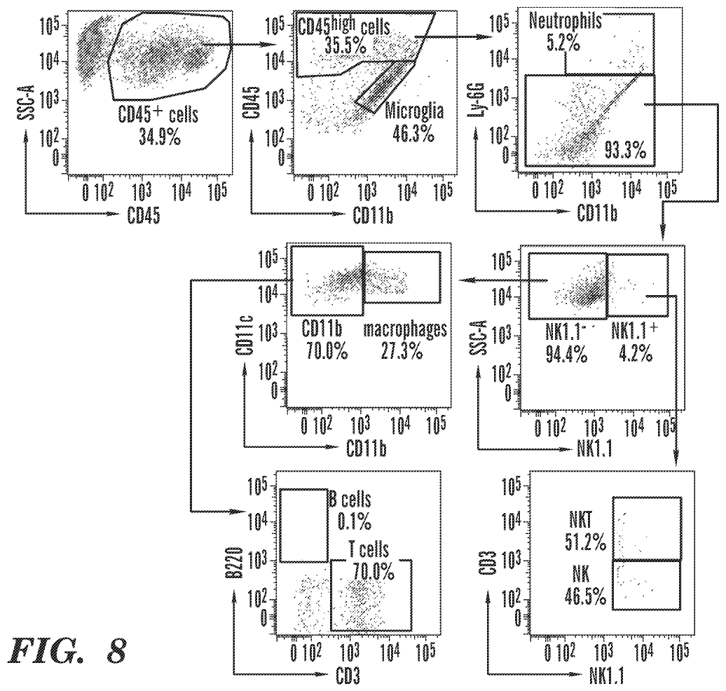

The flow cytometry gating strategy utilized for the analyses shown in FIG. 7 is outlined in FIG. 8.

### Example 4—Material and Methods Relating to Examples 5-7 Animal Disease Model

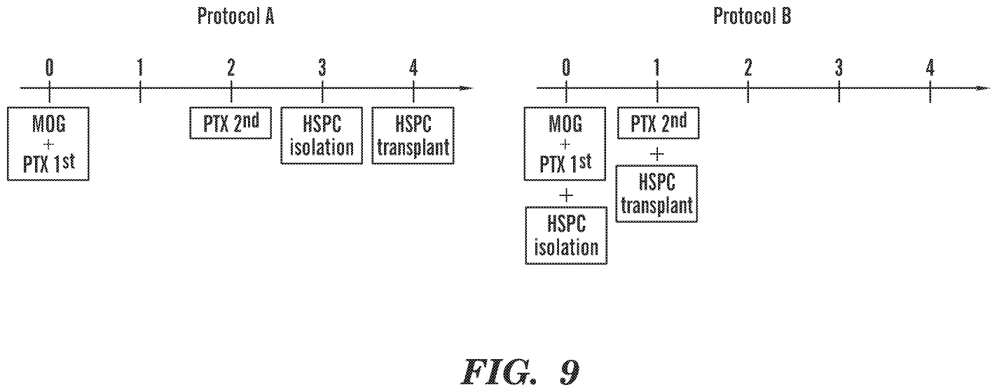

The protocol of immunization was optimized to obtain a disease model characterized by a severe phenotype, a high incidence and ensuring a high reproducibility across experiments. In particular, two different disease induction protocols were optimized to test our therapeutic strategy in different settings. The main differences between the two protocols include the timing of immunization and transplant of hematopoietic stem and progenitor cells (HSPCs). Protocol B is considered less challenging compared to protocol A, because the injection of HSPCs close to immunization could favor a cell-mediated therapeutic reducing the inflammation process. A brief description of both protocols is reported below (FIG. 9).

Protocol (A)

Mice were anaesthetized with isoflurane (4% induction, 2.5% maintenance), and receive at day 0 150 μl of an emulsion containing 200 μg/mouse MOG₃₅₋₅₅ (Espikem, Italy) in incomplete Freund's Adjuvant (IFA, Becton Dickinson, Franklin Lakes, NJ) with 8 mg/ml Mycobacterium tuberculosis (Becton Dickinson), administered via 3 subcutaneous (SC) injections (50 μl each, 1 in each flank and 1 at the base of the tail). In addition, Pertussis toxin (PTX, DBA, Italy) was injected intraperitoneally (IP) (140 μl at 5 ng/μl) on the day of the immunization (day 0) and again after 48 hrs (day 2). Finally, HSPC transplantation was performed at day 4.

Protocol (B)

Mice were anaesthetized with isoflurane (4% induction, 2.5% maintenance), and receive at day 0 150 μl of an emulsion containing 200 μg/mouse MOG₃₅₋₅₅ (Espikem) in IFA (Becton Dickinson) with 8 mg/ml Mycobacterium tuberculosis (Becton Dickinson), administered via 3 SC injections (50 μl each, 1 in each flank and 1 at the base of the tail). In addition, PTX (DBA) was injected IP (140 μl at 5 ng/μl) on the day of the immunization (day 0) and again after 24 hrs (day 1). Finally, HSPC transplantation was performed at day 1, 12 hours after the 2^{nd} PTX injection.

### HSPC Transplantation

HSPCs were isolated from donor C57BL/6 Ly5.1 (CD45.1) mice in order to track in vivo the distribution of donor-derived cells using donor mismatch CD45.1 (donor)/CD45.2 (recipient). Briefly, bone marrow cells were obtained from femurs and tibiae of donor mice by crushing bones with MACS buffer. After lysis, bone marrow cells were lineage-depleted using the Lineage Negative Depletion kit (Miltenyi Biotec, Bergisch Gladbach, Germany) following the manufacturer's instructions. Upon selection, HSPCs were put in culture medium enriched with murine cytokines (IL6, IL3, FLT3, mSCF) for 24 hours and transduced with the optimized lentiviral vector expressing programmed death-ligand 1 (PD-L1) at multiplicity-of-infection 50 to guarantee a high expression of the protein.

Depending on the setting applied, transduced HSPCs (PD-L1 HSPCs) were injected into recipient mice by means of either intravenous (IV), or intracerebroventricular (ICV) or intrathecal lumbar (ITL) route of administration.

In IV transplantation, cells were injected in the tail vein at a dose of 3×10⁶/mouse.

ICV transplantation was performed by surgery, upon anaesthesia with ketamine (100 mg/kg) and xylazine (10 mg/kg). The head of the mouse, shaved and disinfected, was fixed with ear bars in a stereotactic frame and the skin was disclosed longitudinally to visualize the bregma. Under visual control, injection coordinates (1 mm lateral, −0.5 mm anterior) were adjusted to pierce the skull at bregma using a mm diameter drill head. A 10 μl Hamilton syringe was inserted at 2.5 mm depth into the skull and 7 μl cell suspension containing 1×10⁶ Lin⁻ cells was injected. Following wound closure, animals were maintained in sterile conditions.

ITL transplantation was performed under anaesthesia with isoflurane (4% induction, 2.5% maintenance). A 10 μl Hamilton syringe connected to a 30-gauge needle containing 5 μl of cell suspension was introduced into the intervertebral foramen between L5 and L6 through the intact skin. Slow administration of 1×10⁶ cells/mouse was performed once the proper position of the needle was confirmed by tail flick reflex.

### Readouts

The first efficacy readout was the evaluation of the capability of PD-L1 HSPCs to prevent/limit disease manifestations in the optimized EAE model. To this extent, recording of both clinical score and body weight of EAE treated mice was performed daily to monitor the disease course, from disease induction up to the end of the study (30 days post-immunization). The clinical score (0-5) was measured according to the grading system for clinical assessment of EAE (Miller et al. 2007) is presented in Table 1.

At sacrifice, the engraftment and biodistribution of the transplanted cells were monitored in hematopoietic and disease affected organs. In particular, flow cytometry (FC) was used to monitor the quantity and reactivity of inflammatory cells both in the CNS and in peripheral hematopoietic tissues, as readout of the possible immuno-modulatory function exerted by the transplanted cells in the CNS and/or in the periphery. FC data provided information on the engraftment and persistence of transplanted HSPCs in disease-affected organs. To this extent, at day 30 post-immunization mice were euthanized by extensive intracardiac perfusion with cold phosphate-buffered saline (PBS) over 15 min and CNS tissues were then collected and processed as described below.

Brain was removed, and the two hemispheres were differently processed. For immunofluorescence (IF) analysis, one hemisphere was fixed for 24 hours in 4% paraformaldehyde (PFA), embedded in optimal cutting temperature compound and stored at ≤−65° C., after equilibration in sucrose gradients (from 10 to 30%). For FC analysis, brain cells were obtained by mechanic disaggregation of one brain hemisphere in 1.5 ml in Earle's balanced salt solution (EBSS) medium and processed with papain-based digestion procedure according to the neural tissue dissociation kit (Miltenyi Biotec). After washing with EBSS medium, the digested suspension was enriched in myeloid cells with Percoll gradient. Cell suspension were then washed with PBS plus 2% FBS, put in blocking solution and stained with specific antibodies to evaluate the infiltration of inflammatory cells (CD45, Ly6G, CD11b, CD11c, NK1.1; CD3 APC, B220) and to characterize the CD3⁺ population (CD45, CD3, CD4, CD8).

Spinal cord was removed and divided in sections (cervical, thoracic, lumbar). The lumbar and cervical sections were fixed in PFA 4% for IF staining. IF of spinal cords from EAE animals were performed on 10 μm-thick cryosections incubated for 20 minutes with the FluoroMyelin™ Red fluorescent myelin stain (Thermo Fisher, Waltham, MA) in PBS at room temperature. The sections were then washed with PBS, stained with DAPI, mounted and analyzed at confocal microscopy. The remaining tissue was immediately processed for FC, as described for the brain tissue.

### Example 5—Transplantation of PD-L1 HSPCs Modulate the Clinical Course of Immunized and Treated Animals

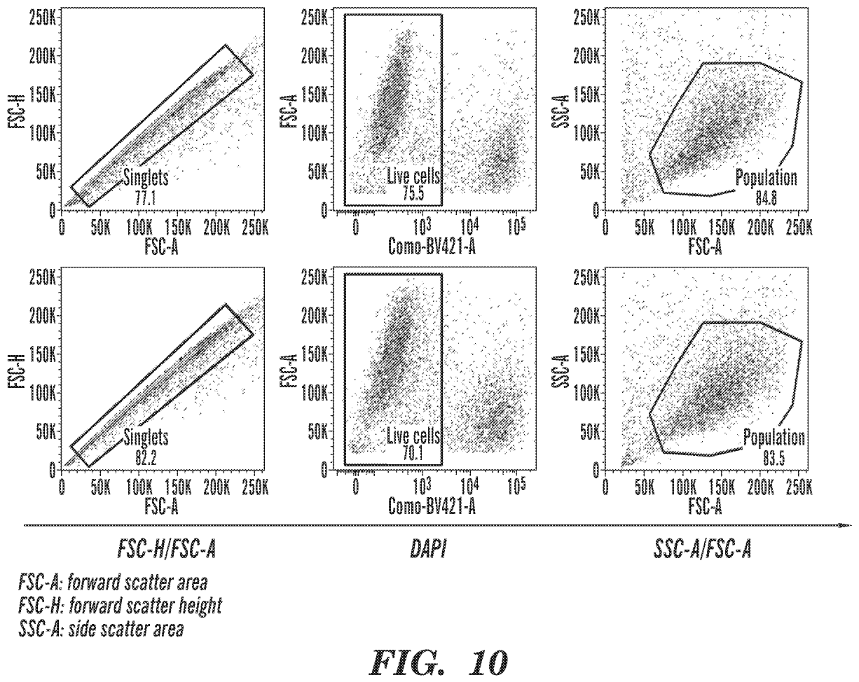

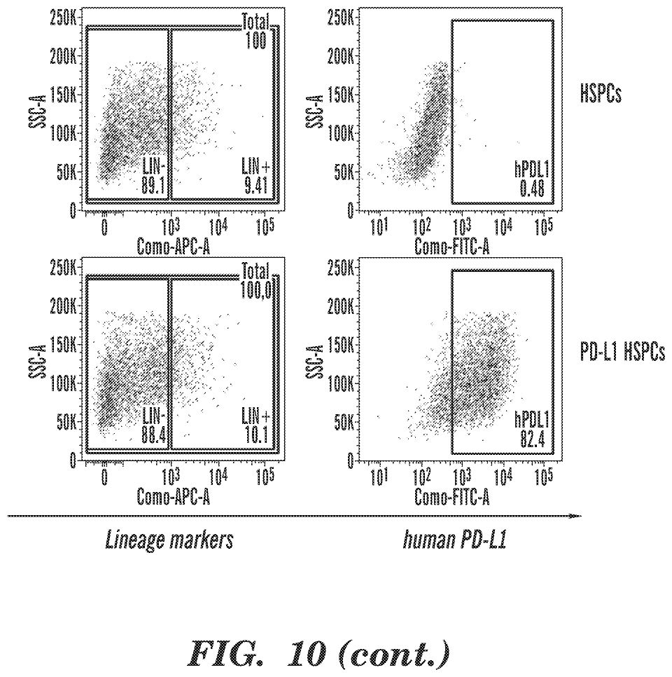

According to Protocol B (FIG. 9), mice underwent MOG₃₅₋₅₅ immunization at day 0 and received PTX on the same day and again after 48 hours (day 2). Mice were injected IV with 3×10⁶ lentiviral vector (LV)-transduced PD-L1-expressing HSPCs at day 4 or were left untreated as control of disease. Before the transplant, FC analysis assessed the quality of cells in terms of vitality and purity of HSPC isolation and confirmed the increase of PD-L1 expression after LV-transduction, as shown in representative plots in FIG. 10.

All C57BL/6 mice were followed-up daily until day 30 (end of study). Independently from the transplant, all MOG₃₅₋₅₅-treated groups developed progressive motor deficits already manifest at day 9 post-immunization and increasing progressively in severity. Body weight loss well correlated with the clinical score measured in animals. Negligible differences were observed in terms of disease onset and maximum disease severity score in all groups, indeed attaining a score ≥3 in all MOG₃₅₋₅₅-treated EAE groups.

Interestingly, a significantly different disease time course was demonstrated in transplanted animals vs. untreated (UT) animals after the peak of disease, with a faster recovery being observed in the treated vs UT mice conceivably mediated by genetically engineered HSPCs expressing PD-L1.

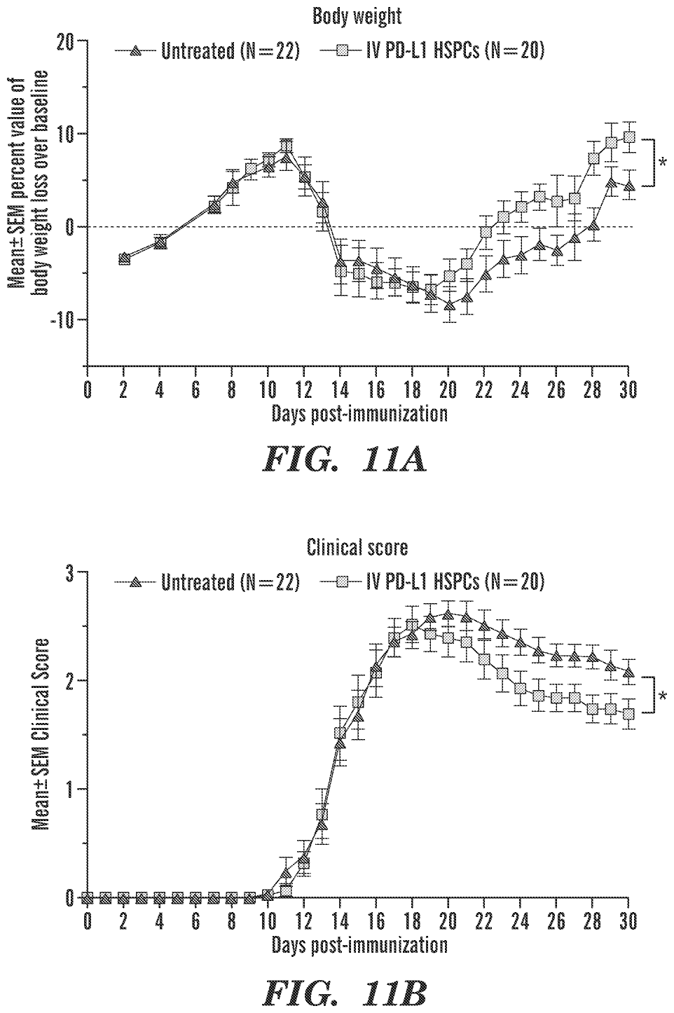

Consistently, body weight showed a constant decrease in all groups until the peak of disease, after which weight values came back to normal most rapidly in mice treated with PD-L1 HSPCs (FIG. 11A).

### Example 6—Transplantation of PD-L1 HSPCs Modulate the CNS Myeloid and T Cell Composition of Immunized and Treated Animals

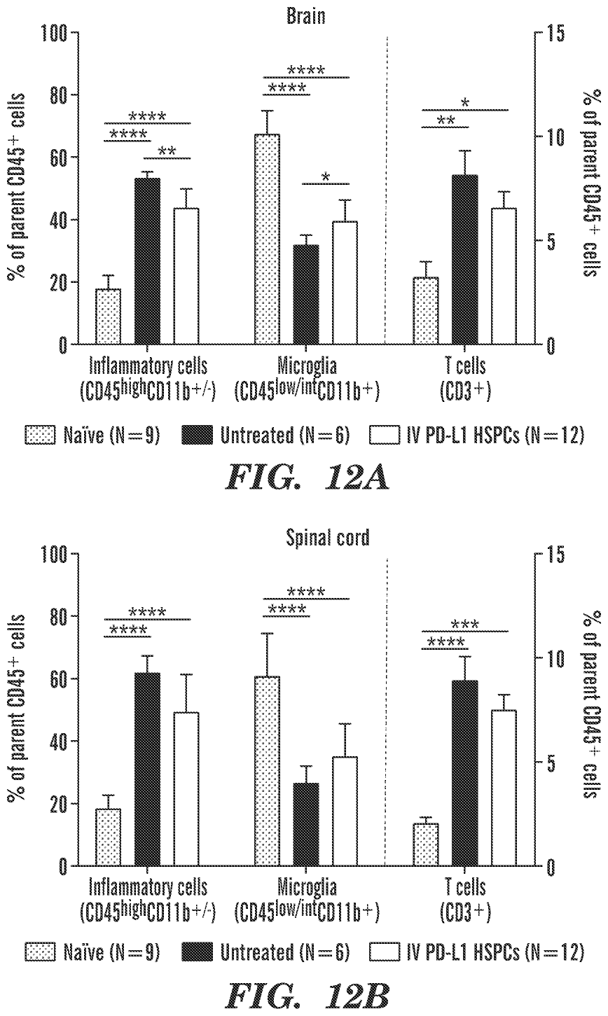

To assess whether the clinical score correlates with the level of inflammation retrieved in the CNS, analysis of myeloid and leukocyte subpopulations was performed in the brain and spinal cord of both naïve and MOG₃₅₋₅₅-immunized animals, the latter either receiving or not receiving PD-L1 HSPCs at day 4. The analysis on myeloid CNS subpopulations, based on the expression of CD45 and CD11b, showed an increase in the proportion of CD45^{high}CD11b^{+/-} cells and a parallel decrease in microglia cells (CD45^{low/int}CD11b⁺ cells) in both brain and spinal cord of EAE MOG₃₅₋₅₅-immunized mice (FIGS. 12A and 12B). These results indicate that there is an expansion of inflammatory cells in the affected regions as a response to disease induction, consistently with the clinical phenotype observed in immunized animals. However, as compared to untransplanted immunized animals, a decrease in the frequency of T cells, i.e., CD3⁺ cells within the total CD45⁺ hematopoietic compartment, was observed in both brain and spinal cord in the PD-L1 HSPCs group. Being that EAE is a T-cell mediated disease, the lower frequency of T cells retrieved in the CNS of animals treated with PD-L1 HSPCs may be the result of a modulatory effect mediated by the progeny of the transplanted cells that in turn promotes a faster recovery, as clinically observed after the peak of disease.

Thus, EAE mice treated with PD-L1 HSPCs undergo a modulation of the immune response at the T cell level, conceivably associated with a faster disease recovery.

### Example 7—Local Administration of PD-L1 HSPC Transplantation to CNS Increases Efficacy of Treatment

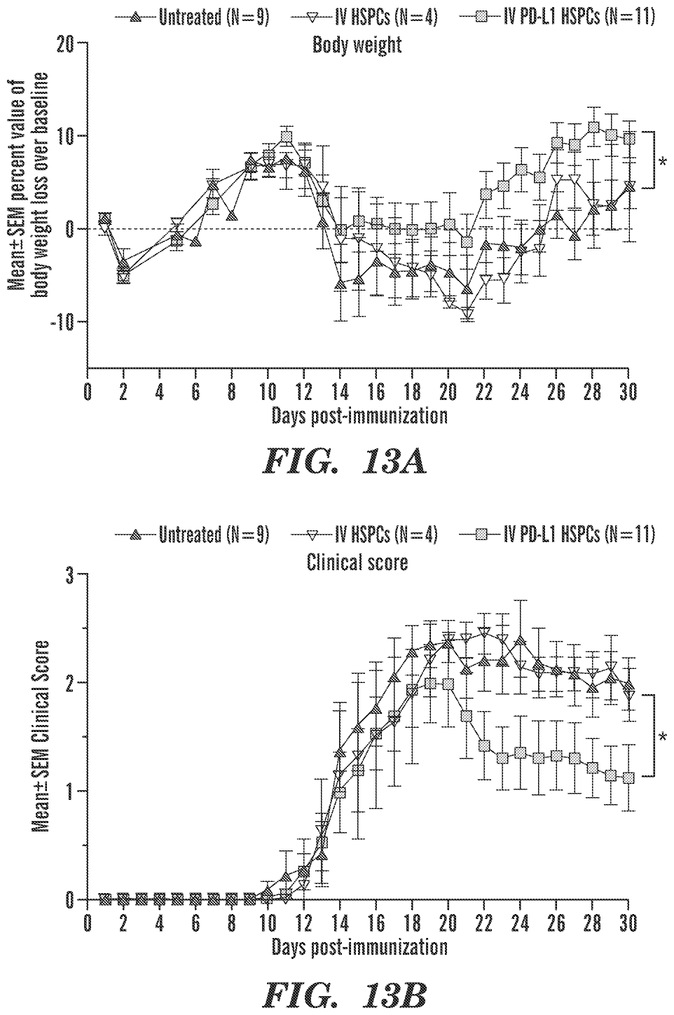

To further investigate and optimize the therapeutic effect of PD-L1-expressing HSPCs for the treatment of MS, the described strategy was tested in an alternative disease setting (Protocol B, FIG. 9), in which the timing between immunization and the transplant has been reduced. As previously mentioned, the rationale is to strengthen the putative immune modulatory effect of the transplanted cells by targeting the early stage of the inflammatory process. To this purpose, animals underwent IV HSPCs transplantation the day after receiving MOG immunization (FIGS. 13A and 13B).

In this new setting, the overall clinical benefit induced by PDL1 HSPCs as compared to disease controls was confirmed, showing a faster recovery after the peak of the disease. Interestingly, the group of mice receiving mock untransduced cells, i.e. untransduced HSPCs, showed a superimposable disease score curve to that of controls (the untreated group). This confirms that the observed clinical benefit is associated with the induced expression of PD-L1 in HSPCs and not to HSPCs alone (FIGS. 13A and 13B).

Using this alternative induction protocol, novel routes of cell delivery directly into the CNS were tested. Without wishing to be bound by a particular theory, it was hypothesized that local delivery into the affected tissues of transduced HSPCs is associated with a greater therapeutic benefit in the EAE model as compared to the conventional IV delivery route. Indeed, direct administration of PD-L1 HSPCs into CNS may contribute to induce a rapid immune-regulatory effect sustained by an early interaction with effector immune cells. Thus, two routes of cell administration were tested alternative to the classic IV route: intracerebroventricular (ICV) administration, and intrathecal lumbar (ITL) administration

Mice undergoing intra CNS HSPC injection received 1×10⁶ PD-L1 HSPCs 12 hours after the last PTX injection, as per Protocol B (FIG. 9). MOG-treated UT control mice underwent the same procedure and were injected with PBS, to prevent any confounding effect associated to the manipulation.

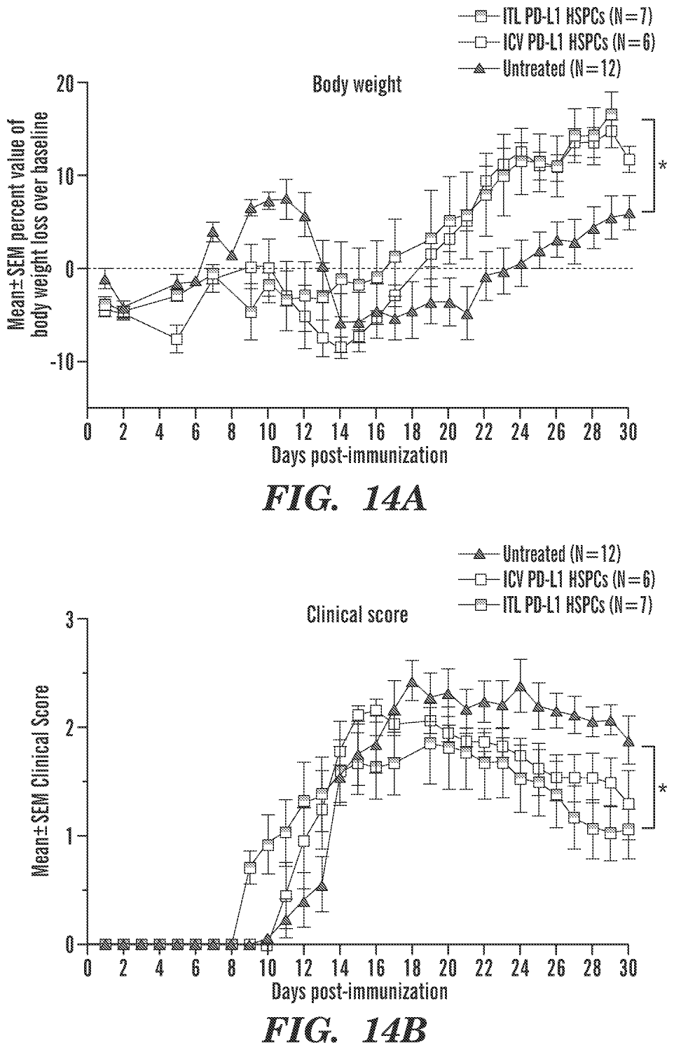

All mice showed disease onset within the expected time window regardless of treatment, indicating that the procedure per se does not affect the disease course (FIGS. 14A and 14B). Notably, an earlier disease onset in the ITL PDL1 HSPC group was observed, possibly related to an early interaction of the transplanted cells with infiltrating inflammatory cells in the CNS. Most importantly, both CNS-transplanted groups (ITL and ICV) showed a milder clinical course as compared to disease controls, characterized by an overall significantly lower mean clinical score along with a faster recovery after the score peak. Consistently, both ITL and ICV PDL1 HSPC animal groups showed the least reduction in body weight post-immunization (FIGS. 14A and 14B).

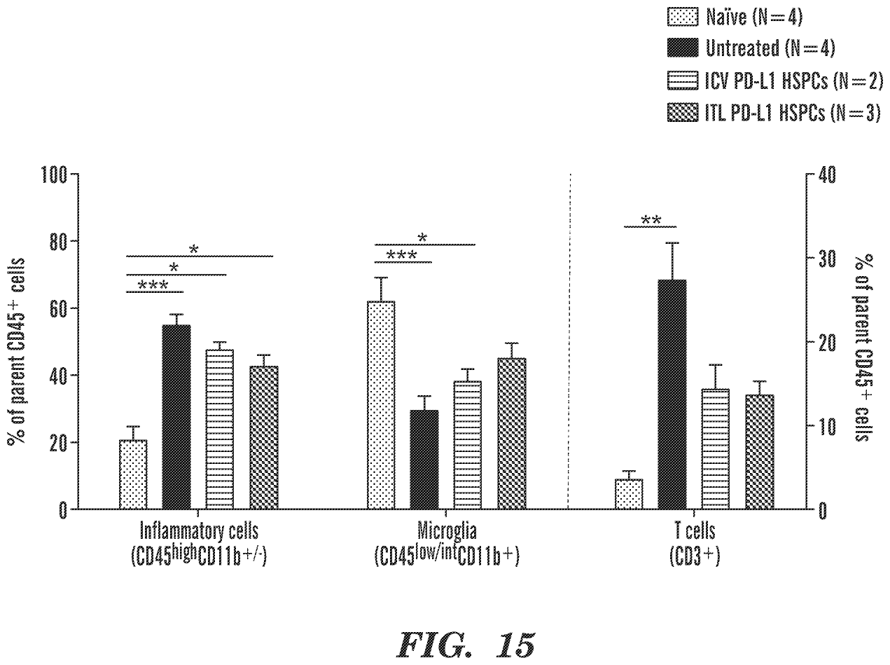

Interestingly, the better clinical outcome observed in mice undergoing direct HSPC administration into the CNS, correlated with an overall lower CNS inflammation. Indeed, both ITL and ICV PD-L1 HSPC treated animals, showed the lowest frequency of inflammatory CD45^{high}CD11b^{+/-} cells in the brain as compared to untreated animals. Likewise, treatment with PD-L1 HSPCs injected either ITL or ICV was associated to the lowest infiltration of CD3⁺ T cells, indicating that PD-L1 HSPCs directly administered into the CNS exert a more effective immuno-regulatory activity aimed at controlling the disease (FIG. 15).

Overall, these data indicate the benefit of administering PD-L1 HSPCs directly into the CNS, specifically the ITL route, which is commonly used in clinical practice.

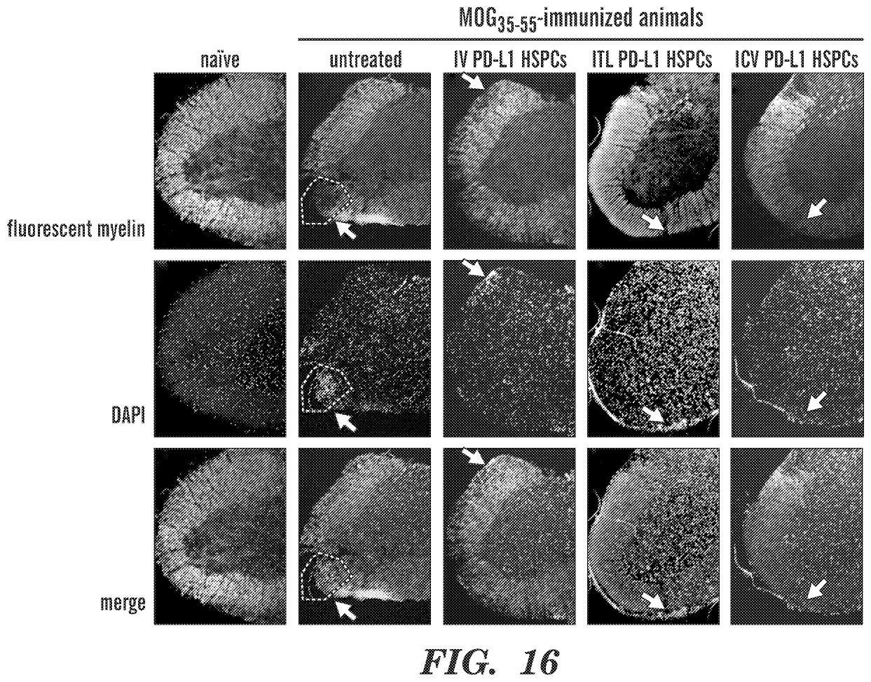

To further assess the activity of PD-L1 HSPCs in the EAE model along with the effect of the route of administration, including the IV, ICV and ITL routes, the extent of demyelination in spinal cord coronal sections at day 30 were analyzed from naïve and MOG₃₅₋₅₅-immunized mice, the latter including untreated animals and mice receiving either untransduced HSPCs or PD-L1 HSPCs via the IV, ICV or ITL routes of administration (FIG. 16).

As expected, MOG₃₅₋₅₅-immunization induced severe demyelination along with a remarkable increase in cell density in untreated mice as compared to naïve animals, as shown by myelin (FluoroMyelin) and nuclear (DAPI) staining (FIG. 16). The higher cell density in correspondence of areas with loss of myelin indicates the recruitment of infiltrating cells at the level of the lesion. Interestingly, mice treated with IV PD-L1 HSPCs showed a milder pattern as compared to untreated mice, with a remarkable reduction of demyelination areas (FIG. 16). Most importantly, analysis of sections from MOG₃₅₋₅₅-immunized animals receiving PD-L1 HSPCs via either ICV or ITL delivery indicates an even greater therapeutic effect as compared to what untreated and IV PD-L-1 HSPC-treated animals, showing only very restricted areas of demyelination and low cellularity, mostly confined closed to the meninges (FIG. 16). In conclusion, the significant reduction of demyelination in EAE mice treated with PD-L1 HSPCs correlates with the positive clinical effect, as assessed by changes in the clinical score, observed after the peak of the disease (FIGS. 13A, 13B, 14A and 14B).

1. A method of treating a central nervous system (CNS) disease or disorder, the method comprising administering to a subject diagnosed with the CNS disease or disorder a composition comprising a population of genetically modified, programmed cell death-1 receptor ligand (PD-L1)⁺-expressing hematopoietic stem cells (HSCs),
wherein the CNS disease or disorder involves inflammation of the CNS.

2. The method of claim 1, wherein the CNS disease or disorder is selected from the group consisting of multiple sclerosis (MS), Systemic lupus erythematosus (SLE), inflammatory brain disease, inflammation of the CNS, central nervous system vasculitis, and Neuromyelitis Optica Spectrum Disorder.

3. The method of claim 1, wherein the CNS disease or disorder is MS.

4. The method of claim 3, wherein the MS is relapsing remitting MS (RRMS).

5. The method of claim 3, wherein the MS is secondary progressing MS (SPMS).

6. The method of claim 3, wherein the MS is primary progressive MS (PPMS).

7. The method of any of claims 3-6, wherein the MS is non-active, active, highly active (HA), or rapidly evolving severe relapsing remitting MS (RE).

8. The method of claim 1, wherein the CNS disease or disorder is inflammation of the CNS.

9. The method of claim 8, wherein inflammation of the CNS is inflammation of the spinal cord.

10. The method of claim 8, wherein inflammation of the CNS is inflammation of the brain.

11. The method of claim 8, wherein inflammation of the CNS is inflammation of the spinal cord and brain.

12. The method of claim 1, further comprising the step, prior to administering, of diagnosing the subject as having a CNS disease or disorder.

13. The method of claim 1, further comprising the step, prior to administering, of receiving the results of an assay that diagnoses a subject as having CNS disease or disorder.

14. The method of claim 1, further comprising the step, prior to administering, of diagnosing the subject as having MS.

15. The method of claim 1, further comprising the step, prior to administering, of receiving the results of an assay that diagnoses a subject as having MS.

16. The method of claim 1, further comprising the step, prior to administering, of diagnosing the subject as having inflammation of the CNS.

17. The method of claim 1, further comprising the step, prior to administering, of receiving the results of an assay that diagnoses a subject as having inflammation of the CNS.

18. The method of claim 1, wherein the PD-L1⁺-expressing HSCs carry an exogenous copy of a nucleic acid encoding a programmed cell death-1 receptor ligand (PD-L1).

19. The method of claim 18, wherein the nucleic acid is a complementary DNA (cDNA).

20. The method of claim 19, wherein the cDNA has the sequence of SEQ ID NO: 1.

21. The method of claim 19, wherein the cDNA has the sequence of SEQ ID NO: 2.

22. The method of claim 18, wherein the nucleic acid is a genomic DNA.

23. The method of any of claim 18-22, wherein the nucleic acid is integrated into the genome of the cells.

24. The method of any of claim 18-23, wherein the nucleic acid has been introduced into the cells via a vector.

25. The method of claim 24, wherein the vector is a viral vector or non-viral vector.

26. The method of claim 25, wherein the viral vector is a lentiviral vector.

27. The method of claim 1, wherein the PD-L1⁺-expressing HSCs are mammalian HSCs or human HSCs.

28. The method of claim 1, wherein the PD-L1⁺-expressing HSCs are obtained by genetically modifying HSCs obtained from bone marrow, umbilical cord, amniotic fluid, chorionic villi, cord blood, placental blood or peripheral blood.

29. The method of claim 1, wherein the PD-L1⁺-expressing HSCs are obtained by genetically modifying HSCs obtained from mobilized peripheral blood.

30. The method of claim 1, further comprising the step, prior to administering, of obtaining HSCs from bone marrow, umbilical cord, amniotic fluid, chorionic villi, cord blood, placental blood or peripheral blood, and genetically modifying the obtained HSCs by introducing an exogenous copy of a nucleic acid encoding a PD-L1.

31. The method of claim 30, wherein the obtained HSC cells are ex vivo cultured before, or after, or both before and after the introduction of the exogenous copy of a nucleic acid encoding a PD-L1.

32. The method of any of claims 1, and 27-29, wherein the HSCs are derived from a healthy individual.

33. The method of any of claims 1, and 27-29, wherein the HSCs are derived from an individual with a diagnosed disease or disorder.

34. The method of claim 33, wherein the diagnosed disease or disorder is a CNS disease or disorder.

35. The method of claim 34, wherein the CNS disease or disorder is MS.

36. The method of claim 34, wherein the CNS disease or disorder is inflammation of the CNS.

37. The method of any of claims 1, and 27-29, wherein the HSCs are derived from the subject.

38. The method of claim 1, wherein the population of PD-L1⁺-expressing HSCs are autologous, allogeneic, or xenogeneic to the subject.

39. The method of claim 1, wherein the PD-L1⁺-expressing HSCs are produced by a method comprising:
d) contacting a population of unmodified HSCs with a vector carrying an exogenous copy of a nucleic acid encoding a PD-L1, thereby obtaining modified HSCs;
e) ex vivo culturing the resultant modified cells from the contacting; and
f) establishing expression of PD-L1 on the modified HSCs, thereby producing a population of modified HSCs cells expressing PD-L1.

40. The method of claim 39, wherein the method further comprises establishing that the population of modified HSCs have an at least 1-fold, 2-fold, 3-fold, 4-fold, 5-fold or more percent increase in the number of PD-L1 expressing HSCs compared to the population of unmodified HSCs.

41. The method of any of claims 1-40, wherein administration is systemic.

42. The method of any of claims 1-40, wherein administration is local administration to at least a lesion, the brain, or the spinal cord.

43. The method of claim 42, wherein the lesion is a site of nerve cell damage.

44. The method of claim 42 or 43, wherein the lesion is present on the brain or spinal cord.

45. The method of any of claim 1-44, wherein administering reduces, delays, or stops the progression of MS.

46. The method of any of claim 1-44, wherein administering reduces or eliminates inflammation of the CNS.

47. The method of any of claim 1-44, wherein administering reduces or eliminates the population of neutrophils in the CNS.

48. The method of any of claim 1-44, wherein administering increases the population of CD3+ T cells in the CNS.

49. The method of any of claims 1-44, wherein the PD-L1⁺-expressing HSCs are detected at lesions in the subject following administration.

50. The method of any of claims 1-49, further comprising administering at least one additional therapeutic.

51. The method of claim 50, wherein the at least one additional therapeutic is an anti-MS therapeutic.

52. A method of treating a CNS disease or disorder associated with inflammation of the CNS in a subject comprising:
a) providing a population of unmodified hematopoietic stem cells (HSCs);
b) contacting the population of unmodified HSCs of (a) with a vector carrying an exogenous copy of a nucleic acid encoding a PD-L1, thereby obtaining modified HSCs;
c) ex vivo culturing the resultant modified HSCs from the contacting;
d) establishing expression of PD-L1 on the modified HSCs, thereby producing a population of PD-L1⁺-expressing HSCs; and
e) transplanting said population of PD-L1⁺-expressing HSCs into a recipient subject having a CNS disease or disorder associated with inflammation of the CNS, thereby treating the CNS disease or disorder in the recipient subject.

53. A method of treating MS in a subject comprising:
a) providing a population of unmodified hematopoietic stem cells (HSCs);
b) contacting the population of unmodified HSCs of (a) with a vector carrying an exogenous copy of a nucleic acid encoding a PD-L1, thereby obtaining modified HSCs;
c) ex vivo culturing the resultant modified HSCs from the contacting;
d) establishing the expression of PD-L1 on the modified HSCs, thereby producing a population of PD-L1⁺-expressing HSCs; and
e) transplanting said population of PD-L1⁺-expressing HSCs into a recipient subject having MS, thereby treating MS in the recipient subject.

54. A method of treating or preventing inflammation of the CNS in a subject comprising:
a) providing a population of unmodified hematopoietic stem cells (HSCs);
b) contacting the population of unmodified HSCs of (a) with a vector carrying an exogenous copy of a nucleic acid encoding a PD-L1, thereby obtaining modified HSCs;
c) ex vivo culturing the resultant modified HSCs from the contacting;
d) establishing expression of PD-L1 on the modified HSCs, thereby producing a population of PD-L1⁺-expressing HSCs; and
e) transplanting said population of PD-L1⁺-expressing HSCs into a recipient subject having or at risk of having inflammation of the CNS, thereby preventing or treating inflammation of the CNS in the recipient subject.

55. A method of treating a CNS disease or disorder associated with inflammation of the CNS in a subject comprising:
a) receiving a population of PD-L1⁺-expressing HSCs; and
b) transplanting said population of PD-L1⁺-expressing HSCs into a recipient subject having a CNS disease or disorder associated with inflammation of the CNS, thereby treating the CNS disease or disorder in the recipient subject.

56. A method of treating MS in a subject comprising:
a) receiving a population of PD-L1⁺-expressing HSCs; and
b) transplanting said population of PD-L1⁺-expressing HSCs into a recipient subject having MS, thereby treating MS in the recipient subject.

57. A method of treating or preventing inflammation of the CNS in a subject comprising:
a) receiving a population of PD-L1⁺-expressing HSCs; and
b) transplanting said population of PD-L1⁺-expressing HSCs into a recipient subject having or at risk of having inflammation of the CNS, thereby preventing or treating inflammation of the CNS in the recipient subject.

58. The method of any of claims 52-57, wherein the population of HSCs is autologous to the recipient subject.

59. The method of any of claims 52-57, wherein the population of HSCs is allogeneic to the recipient subject.

60. The method of any of claims 52-57, wherein the population of HSCs is xenogeneic to the recipient subject.

61. A population of PD-L1⁺-expressing HSCs, comprising HSCs obtained from a subject diagnosed with a CNS disease or disorder and having an exogenous copy of a nucleic acid encoding PD-L1.

62. The population of PD-L1⁺-expressing HSCs of claim 61, wherein the exogenous copy of the nucleic acid encoding PD-L1 is integrated into the genome of the HSCs.

63. The population of PD-L1⁺-expressing HSCs of claim 61, wherein the CNS disease or disorder is MS.

64. A pharmaceutical composition comprising the population of PD-L1⁺-expressing HSCs of any one of claims 61-63 in a physiologically acceptable excipient.

65. An ex vivo method of producing a population of PD-L1⁺-expressing hematopoietic stem cells (HSCs), the method comprising:
a) obtaining a population of unmodified HSCs from a subject diagnosed with a CNS disease or disorder;
b) contacting the population of unmodified HSCs with a vector carrying an exogenous copy of a nucleic acid encoding PD-L1, thereby obtaining modified HSCs;
c) ex vivo culturing the resultant modified HSCs; and
d) establishing expression of PD-L1 on the modified HSCs, thereby producing a population of modified HSCs expressing PD-L1.

66. The method of claim 65, wherein in step a), the population of unmodified HSCs are obtained from the bone marrow, umbilical cord, amniotic fluid, chorionic villi, cord blood, placental blood or peripheral blood of the subject.

67. The method of any one of claims 65-66, wherein the CNS disease or disorder is MS.

68. The of any of claims 1-60, wherein administering is intravenous administration, intrathecal administration, or intracerebroventricular administration.


SEQUENCE LISTING (4 sequences)

SEQ ID NO: 1 - DNA, 531 bp, Homo sapiens
atgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 2 - DNA, 873 bp, Homo sapiens
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 3 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET